(19) United States
(12) Patent Application Publication
(10) Pub. No.: US 2009/0137613 A1
(43) Pub. Date: May 28, 2009

(54) PHARMACEUTICALLY ACCEPTABLE SALTS AND POLYMORPHIC FORMS

(76) Inventors: Helena Ceric, Zagreb (HR); Zvonimir Siljkovic, Zagreb (HR); Iva Tunjic, Zagreb (HR); Aleksandar Danilovski, Zagreb (HR); Ernest Mestrovic, Zagreb (HR)

Correspondence Address: FOLEY & LARDNER LLP, 150 EAST GILMAN STREET, P.O. BOX 1497, MADISON, WI, 53701-1497, US

(21) Appl. No.: 12/293,239

(22) PCT Filed: Mar. 6, 2007
(86) PCT No.: PCT/GB07/00764

§ 371 Date: Jan. 8, 2009

(30) Foreign Application Priority Data
Mar. 17, 2006 (GB) 0605344.1

(51) Int. Cl. A61K 31/522

(52) U.S. Cl. 514/263.35

(57) ABSTRACT

The present invention is concerned with new pharmaceutically acceptable salts of valacyclovir, polymorphic forms, processes for preparing the new pharmaceutically acceptable salts and new polymorphic forms, pharmaceutical compositions containing the same, therapeutic uses thereof and methods of treatment employing the same.

The present invention is concerned with new pharmaceutically acceptable salts of valacyclovir and new polymorphic forms processes for preparing the new pharmaceutically acceptable salts and new polymorphic forms, pharmaceutical compositions containing the same, therapeutic uses thereof and methods of treatment employing the same.

Valacyclovir is an L-valyl ester prodrug of acyclovir, being rapidly and almost completely converted in vivo by first-pass metabolism to acyclovir, probably by the enzyme referred to as valacyclovir hydrolase.

Acyclovir is chemically designated as 9-[(2-hydroxyethoxy)methyl]guanine and can be represented by the following structural formula:

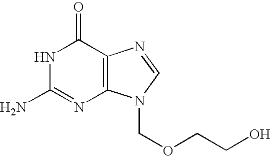

Acyclovir is an acyclic guanine nucleoside analogue which has been found to have potent anti-viral activity and is widely used in the treatment and prophylaxis of viral infections, particularly infections caused by the herpes group of viruses.

Acyclovir inhibits viral DNA synthesis once it has been phosphorylated to the active triphosphate form. The first stage of phosphorylation, to the monophosphate, requires the activity of a virus-specific enzyme. This requirement for activation of acyclovir by a virus-specific enzyme largely explains its selectivity. The phosphorylation process is completed (conversion from mono- to triphosphate) by cellular kinases. Acyclovir triphosphate competitively inhibits the virus DNA polymerase and incorporation of this nucleoside analogue results in obligate chain termination, halting virus DNA synthesis and thus blocking virus replication.

The herpes group of viruses includes herpes simplex virus types I and II, varicella zoster virus, cytomegalovirus, Epstein-Barr virus and human herpes virus 6. Some of the diseases caused by herpes viruses are cold sores, genital herpes, herpes keratitis, herpes encephalitis, chickenpox, shingles, post-herpetic neuralgia, infectious mononucleosis, Burkitt's lymphoma, cytomegaloviral retinitis, roseola and Kaposi's sarcoma.

Acyclovir is, however, poorly absorbed from the gastrointestinal tract after oral administration and this low bioavailability means that multiple large doses of drug may need to be administered in order to achieve and maintain effective anti-viral levels in plasma. This is particularly important in the treatment of infections caused by those viruses which are more resistant to the drug.

Valacyclovir is chemically designated as L-valine 2-[(2-amino-1,6-dihydro-6-oxo-9H-purin-9-yl)methoxy]ethyl ester and can be represented by the following structural formula:

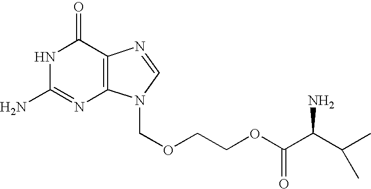

In comparison to acyclovir, valacyclovir provides improved bioavailability. This is because it has been shown to be rapidly absorbed from the gastrointestinal tract after oral administration.

The basic NCE patent for valacyclovir is EP 0 308 065B. Example 1A relates to the preparation of valacyclovir as free base and Example 1B relates to the preparation of valacyclovir hydrochloride monohydrate. The only enabling disclosure of a salt of valacyclovir in EP 0 308 065B is of valacyclovir hydrochloride monohydrate.

EP 0 804 436B discloses an anhydrous crystalline form of valacyclovir hydrochloride.

EP 1 436 295A discloses various polymorphic crystalline forms of valacyclovir hydrochloride which are designated Forms I and II and IV-VII.

EP 1 453 834A discloses an anhydrous polymorphic crystalline form of valacyclovir hydrochloride.

EP 1 575 953A discloses an anhydrous polymorphic crystalline form of valacyclovir hydrochloride.

WO 04106338A discloses various polymorphic crystalline forms of valacyclovir hydrochloride which are designated Forms VIII-XIV.

WO 05000850A discloses various polymorphic crystalline forms of valacyclovir hydrochloride which are designated Forms V and VIII-XII.

WO 05085247A discloses various polymorphic crystalline forms of valacyclovir hydrochloride which are designated Forms I, II, IV, VI and VII.

Valacyclovir hydrochloride has been commercially developed by GlaxoSmithKline and is available under the trademark Valtrex. It has been found that valacyclovir hydrochloride is moderately soluble in water.

It is well recognised in the pharmaceutical field that the provision of a drug in a form that is poorly or moderately soluble in water can result in less than optimal performance and thus the provision of a drug form with enhanced solubility is desirable. Poorly or moderately soluble drugs often exhibit incomplete or erratic absorption and hence low bioavailability and slow onset of action. The effectiveness of poorly or moderately soluble drugs can vary from patient to patient, and there can be a strong effect of food on the absorption of such drugs. For certain poorly soluble drugs it has been necessary to increase the dose thereof to obtain the efficacy required.

Polymorphic forms of a drug substance can have different chemical and physical properties, including melting point, chemical reactivity, apparent solubility, dissolution rate, optical and mechanical properties, vapor pressure, and density. These properties can have a direct effect on the ability to process and/or manufacture a drug substance and a drug product, as well as on drug product stability, dissolution, and bioavailability. Thus, polymorphism can affect the quality, safety, and efficacy of a drug product.

Polymorphic forms as referred to herein can include crystalline and amorphous forms as well as solvate and hydrate forms, which can be further characterised as follows:

(i) Crystalline forms have different arrangements and/or conformations of the molecules in the crystal lattice.

(ii) Amorphous forms consist of disordered arrangements of molecules that do not possess a distinguishable crystal lattice.

(iii) Solvates are crystal forms containing either stoichiometric or non-stoichiometric amounts of a solvent. If the incorporated solvent is water, the solvate is commonly known as a hydrate.

When a drug substance exists in polymorphic forms, it is said to exhibit polymorphism.

There are a number of methods that can be used to characterise polymorphs of a drug substance. Demonstration of a non-equivalent structure by single crystal X-ray diffraction is currently regarded as the definitive evidence of polymorphism. X-ray powder diffraction can also be used to support the existence of polymorphs. Other methods, including microscopy, thermal analysis (e.g., differential scanning calorimetry, thermal gravimetric analysis, and hot-stage microscopy), and spectroscopy (e.g., infrared (IR) and near infrared (NIR), Raman and solid-state nuclear magnetic resonance [ssNMR]) are also helpful to further characterise polymorphic forms.

Drug substance polymorphic forms can exhibit different chemical, physical and mechanical properties as referred to above, including aqueous solubility and dissolution rate, hygroscopicity, particle shape, density, flowability, and compactability, which in turn may affect processing of the drug substance and/or manufacturing of the drug product. Polymorphs can also exhibit different stabilities. The most stable polymorphic form of a drug substance is often chosen during drug development based on the minimal potential for conversion to another polymorphic form and on its greater chemical stability. However, a meta-stable form can alternatively be chosen for various reasons, including better bioavailability.

There is now provided by the present invention, therefore, pharmaceutically acceptable salts of valacyclovir with advantageous properties. More specifically, we have now surprisingly found that certain valacyclovir salts exhibit beneficial properties and, in particular, provide advantages over commercially available valacyclovir hydrochloride.

There is now provided by the present invention, therefore, a pharmaceutically acceptable salt of valacyclovir, wherein said salt is formed between valacyclovir free base and a pharmaceutically acceptable acid selected from the group consisting of methanesulphonic acid, phosphoric acid, maleic acid, fumaric acid, tartaric acid and citric acid.

In particular there is provided by the present invention valacyclovir mesylate, valacyclovir phosphate, valacyclovir maleate, valacyclovir fumarate, valacyclovir tartrate and valacyclovir citrate.

Each of the salts provided by the present invention is also characterised herein as one or more novel polymorphic forms and as such there is also provided by the present invention new polymorphic forms of valacyclovir mesylate, valacyclovir phosphate, valacyclovir maleate, valacyclovir fumarate, valacyclovir tartrate and valacyclovir citrate. More particularly, there is provided by the present invention polymorph I of valacyclovir mesylate; polymorphs I, II and III of valacyclovir phosphate; polymorph I of valacyclovir maleate; polymorphs I and II of valacyclovir fumarate; polymorph I of valacyclovir tartrate and polymorph I of valacyclovir citrate.

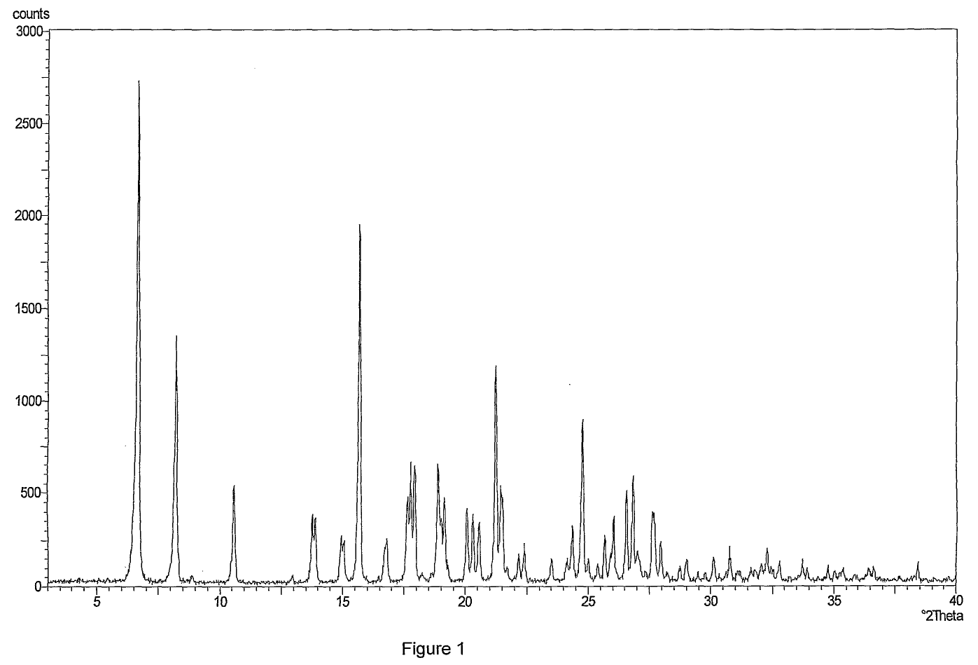

The crystalline structure of polymorph I of valacyclovir mesylate according to the present invention is characterised as having an X-ray powder diffraction pattern, or substantially the same X-ray powder diffraction pattern, as shown in FIG. 1.

Polymorph I of valacyclovir mesylate according to the present invention is further characterised as having characteristic peaks (2θ): 6.69, 8.23, 10.59, 13.76 and 15.68 (±0.2). Further peaks (2θ) associated with polymorph I of valacyclovir mesylate according to the present invention are: 17.93, 18.87, 20.30, 21.22 and 24.76 (±0.2).

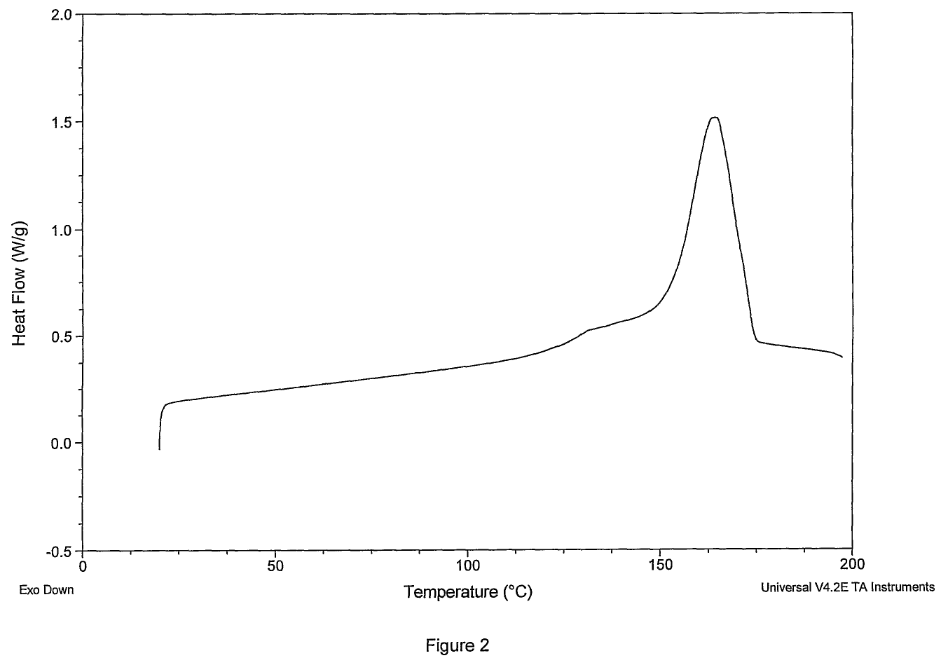

Polymorph I of valacyclovir mesylate according to the present invention is further characterised by a typical DSC thermograph, or substantially the same DSC thermograph, as shown in FIG. 2. Polymorph I of valacyclovir mesylate has a characteristic DSC melting endotherm at about 156° C.

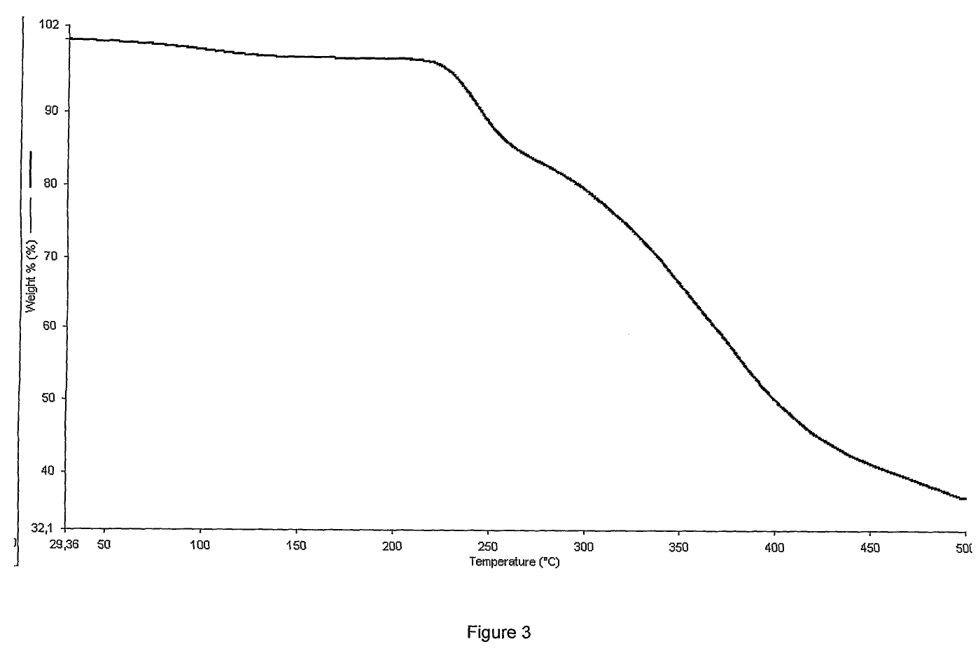

Polymorph I of valacyclovir mesylate according to the present invention is further characterised by a typical TGA thermograph, or substantially the same TGA thermograph, as shown in FIG. 3. As used herein, the term “TGA” refers to thermogravimetric analysis. TGA is a measure of the thermally induced weight loss of a material as a function of the applied temperature. TGA is restricted in transitions that involve either a gain or a loss of mass and it is most commonly used to study desolvation processes and compound decomposition.

Polymorph I of valacyclovir mesylate according to the present invention is further characterised by a TGA weight loss of about 2.5% over the temperature range of about 30-165° C., which confirms that polymorph I of valacyclovir mesylate as prepared according to the present invention is stable to a temperature of about 200° C.

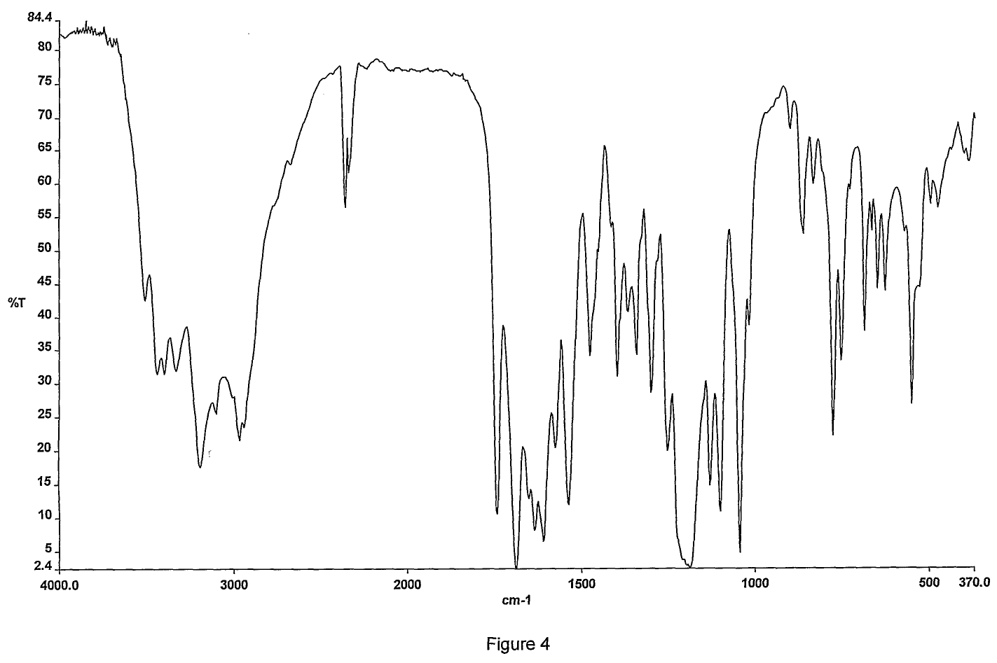

Polymorph I of valacyclovir mesylate according to the present invention is still further characterised as having a Fourier Transform Infrared Spectroscopy (FTIR) pattern, or substantially the same FTIR pattern, as shown in FIG. 4. More particularly, polymorph I of valacyclovir mesylate according to the present invention has characteristic FTIR absorbance bands at about 1746, 1688, 1636, 1538, 1399, 1369, 1189, 1132, 1046, 780, 755, 689, 651 and 553 (±4) cm⁻¹.

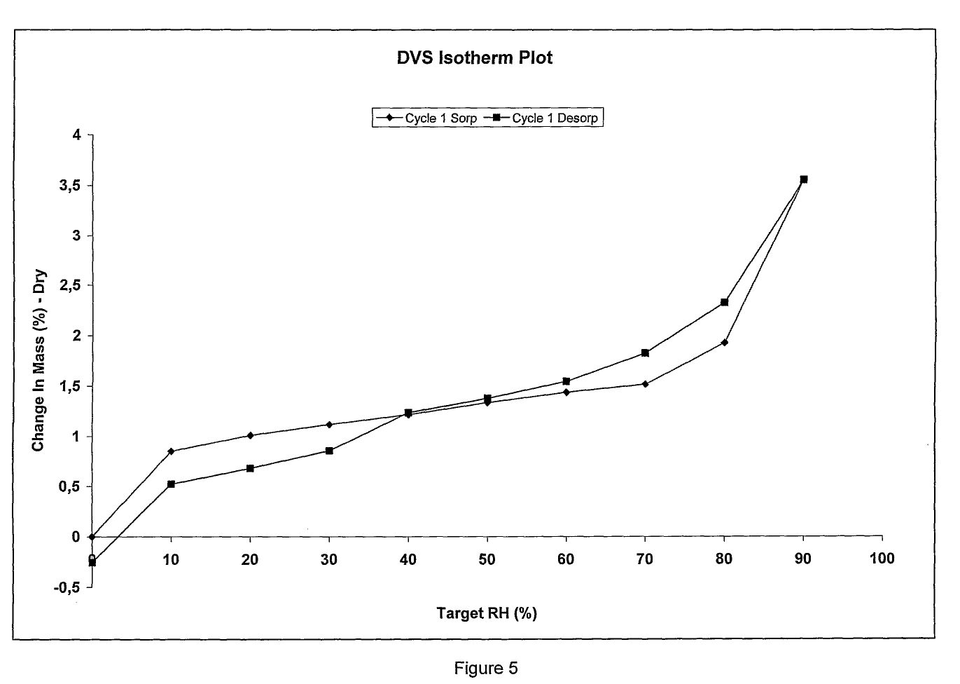

Polymorph I of valacyclovir mesylate according to the present invention can also be characterised by a typical dynamic vapour sorption (DVS) isotherm plot, or substantially the same DVS isotherm plot, as shown in FIG. 5. Polymorph I of valacyclovir mesylate is further characterised by a dynamic vapour sorption (DVS) of about 3.6% at about 90% relative humidity (RH). DVS is a measure of the water vapour or moisture sorption of a material under varying conditions of humidity and it can be used as a measure of the hygroscopicity of a given material.

The water vapour or moisture sorption properties of pharmaceutical materials such as excipients, drug formulations and packaging films are recognized in the art as critical factors in determining the storage, stability, processing and application performance thereof. Moisture sorption properties are thus routinely determined for pharmaceutical materials and have traditionally been evaluated by storing samples over saturated salt solutions of established relative humidities and then regularly weighing until equilibrium is reached. However, there are a number of disadvantages associated with these methods, including: (i) the prolonged period of time taken for the samples to reach equilibrium using a static method, which can often be many days and in many cases can be several weeks; (ii) inherent inaccuracies as the samples have to be removed from the storage container to be weighed, which can cause weight loss or gain; (iii) static methods necessitate the use of large samples sizes (typically >1 gm); and (iv) the highly labour intensive nature of static methods.

The DVS data as described herein was obtained using the Dynamic Vapour Sorption (DVS) methodology developed by Surface Measurement Systems (SMS) Ltd. for the rapid quantitative analysis of the water sorption properties of solids including pharmaceutical materials. The Surface Measurement Systems DVS instrument rapidly measures uptake and loss of moisture by flowing a carrier gas at a specified relative humidity (RH) over a sample (1 mg-1.5 g) suspended from the weighing mechanism of a Cahn D-200 ultra sensitive recording microbalance. This particular microbalance is used because it is capable of measuring changes in sample mass lower than 1 part in 10 million and provides the long-term stability as required for the accurate measurement of vapour sorption phenomena, which may take from minutes to days to complete depending upon the sample size and material. Indeed, a major factor in determining the water sorption behaviour of materials is the need to establish rapid water sorption equilibrium, therefore the DVS instrument allows sorption behaviour to be accurately determined on very small sample sizes (typically 10 mg), thus minimising the equilibration time required.

One of the most critical factors for any instrumentation used for investigating moisture sorption behaviour is the temperature stability of the measurement system. The main DVS instrument systems as used herein are, therefore, housed in a precisely controlled constant temperature incubator with a temperature stability of ±0.1° C. This ensures very good instrument baseline stability as well as accurate control of the relative humidity generation. The required relative humidities are generated by accurately mixing dry and saturated vapour gas flows in the correct proportions using mass flow controllers. Humidity and temperature probes are situated just below the sample and reference holders to give independent verification of system performance. The microbalance mechanism is very sensitive to sorption and desorption of moisture. A constant dry gas purge to the balance head is, therefore, provided to give the best performance in terms of baseline stability. The purge flow is manually controlled such that in the event of a power failure, condensation of moisture in the balance head cannot occur. The DVS instrument is fully automated.

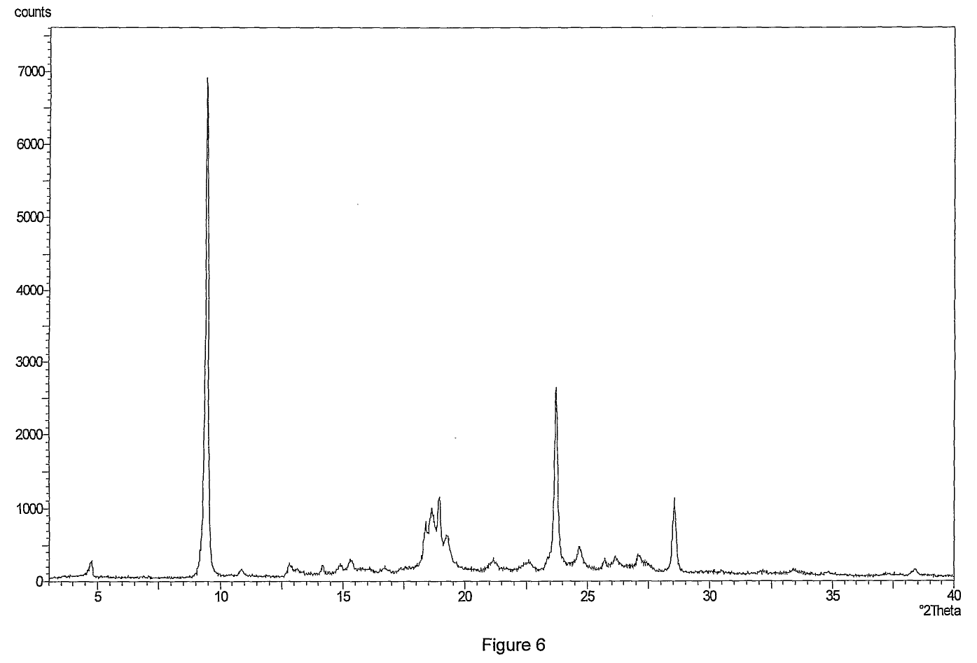

The crystalline structure of polymorph I of valacyclovir phosphate according to the present invention is characterised as having an X-ray powder diffraction pattern, or substantially the same X-ray powder diffraction pattern, as shown in FIG. 6.

Polymorph I of valacyclovir phosphate according to the present invention is further characterised as having characteristic peaks (2θ): 6.87, 8.57, 10.41, 12.96 and 17.16 (±0.2). Further peaks (2θ) associated with polymorph I of valacyclovir phosphate according to the present invention are: 15.28, 15.77, 20.23, 20.87 and 25.47 (±0.2).

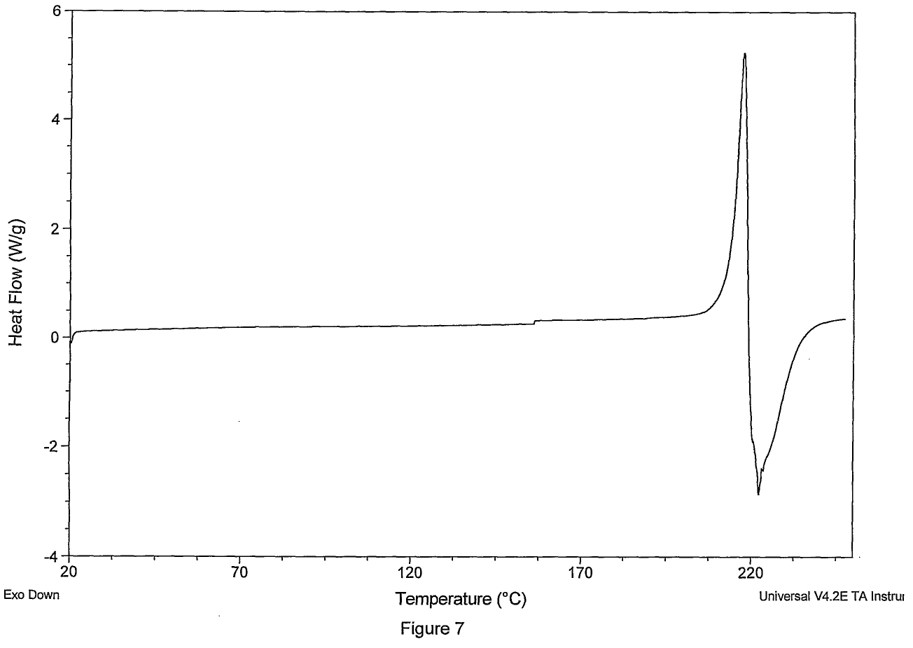

Polymorph I of valacyclovir phosphate according to the present invention is further characterised by a typical DSC thermograph, or substantially the same DSC thermograph, as shown in FIG. 7. Polymorph I of valacyclovir phosphate has a characteristic DSC melting endotherm at about 214° C.

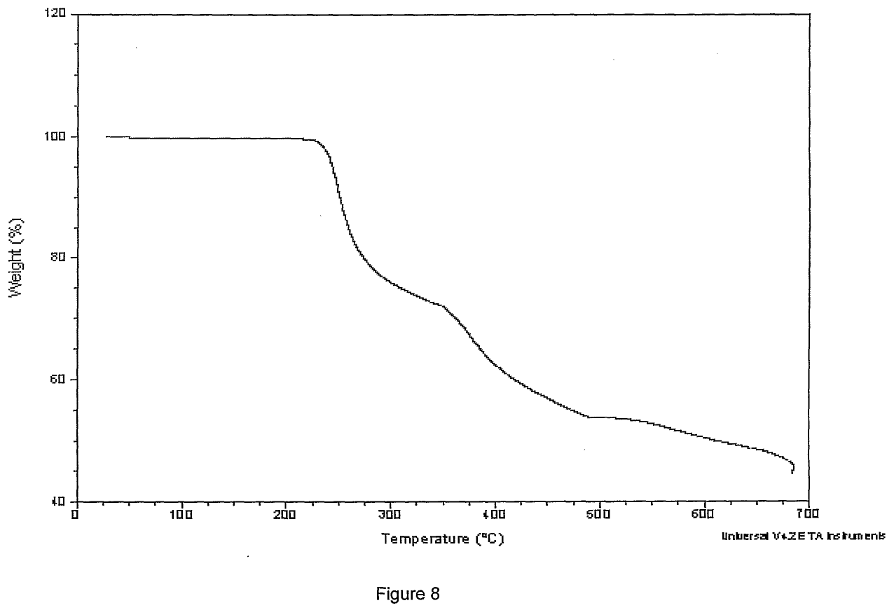

Polymorph I of valacyclovir phosphate according to the present invention is further characterised by a typical TGA thermograph, or substantially the same TGA thermograph, as shown in FIG. 8.

Polymorph I of valacyclovir phosphate according to the present invention is further characterised by no TGA weight loss over the temperature range of about 30-200° C., which confirms that polymorph I of valacyclovir phosphate as prepared according to the present invention is stable to a temperature of about 200° C.

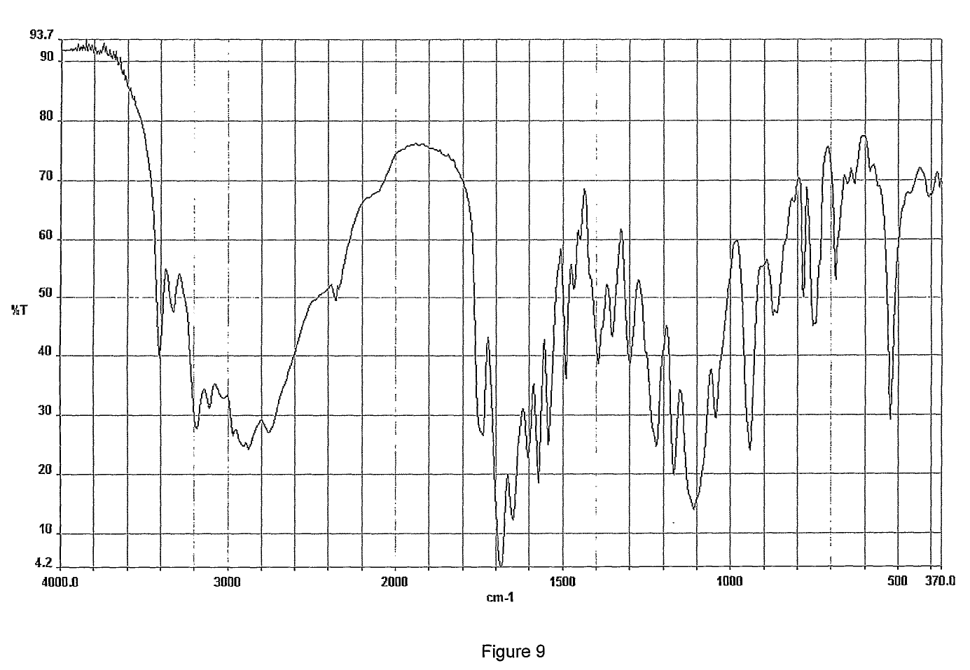

Polymorph I of valacyclovir phosphate according to the present invention is still further characterised as having an FTIR pattern, or substantially the same FTIR pattern, as shown in FIG. 9. More particularly, polymorph I of valacyclovir phosphate according to the present invention has characteristic FTIR absorbance bands at about 1741, 1686, 1651, 1575, 1222, 1170, 1111, 944, 755, 689 and 525 (±4) cm⁻¹.

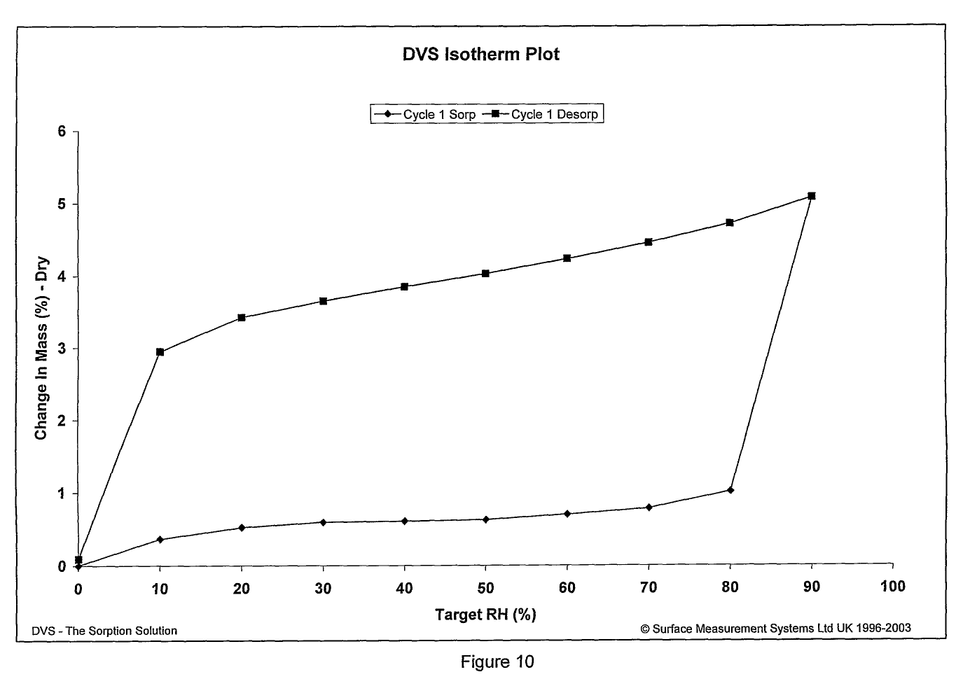

Polymorph I of valacyclovir phosphate according to the present invention can also be characterised by a typical dynamic vapour sorption (DVS) isotherm plot, or substantially the same DVS isotherm plot, as shown in FIG. 10. Polymorph I of valacyclovir phosphate is further characterised by a dynamic vapour sorption of about 1.0% at about 80% RH and about 5.1% at about 90% RH, due to formation of hydrated valacyclovir phosphate form II.

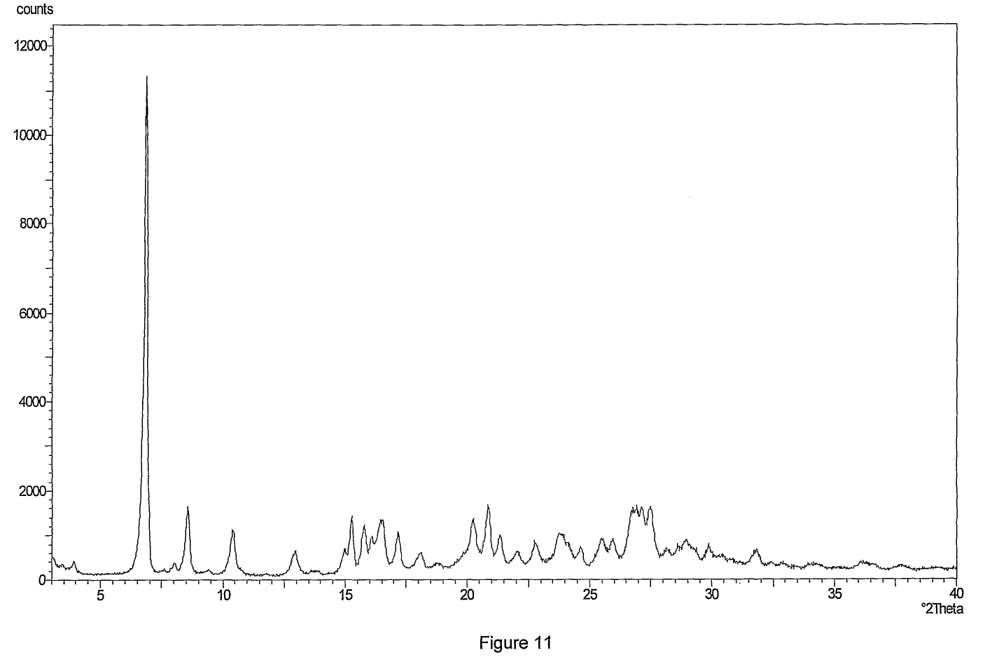

The crystalline structure of polymorph II of valacyclovir phosphate according to the present invention is characterised as having an X-ray powder diffraction pattern, or substantially the same X-ray powder diffraction pattern, as shown in FIG. 11.

Polymorph II of valacyclovir phosphate according to the present invention is further characterised as having characteristic peaks (2θ): 4.75, 9.45, 18.37, 18.61 and 23.71 (±0.2). Further peaks (2θ) associated with polymorph II of valacyclovir phosphate according to the present invention are: 12.79, 18.92, 19.24, 24.66 and 28.55 (±0.2).

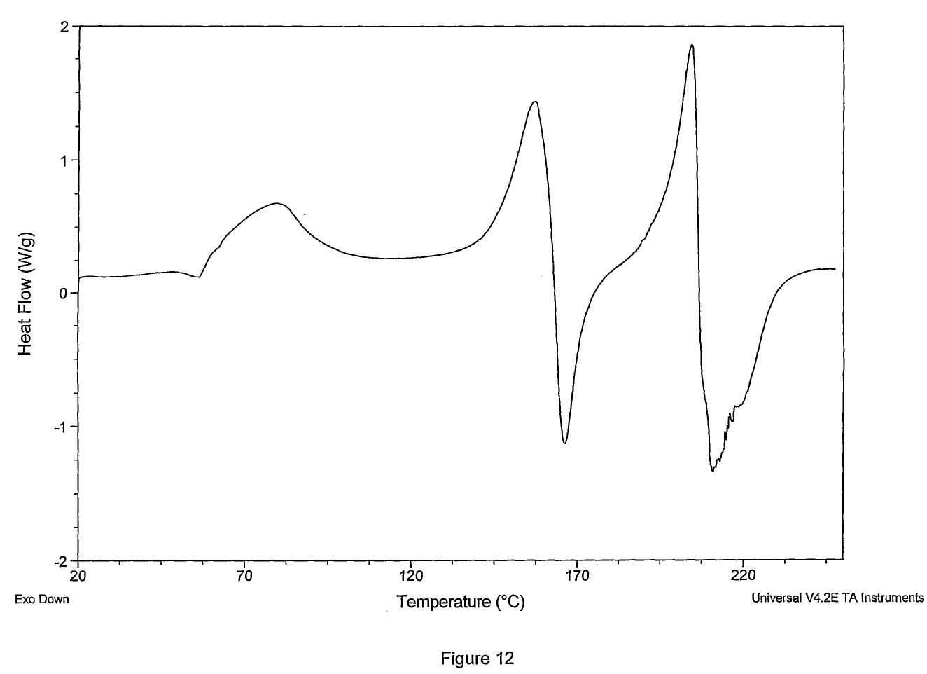

Polymorph II of valacyclovir phosphate according to the present invention is further characterised by a typical DSC thermograph, or substantially the same DSC thermograph, as shown in FIG. 12. Polymorph II of valacyclovir phosphate has a characteristic endotherm in the range of 55-110° C. due to a loss of solvent, a melting endotherm at about 145° C., a recrystallization exotherm at about 163° C. and a melting endotherm at about 196° C.

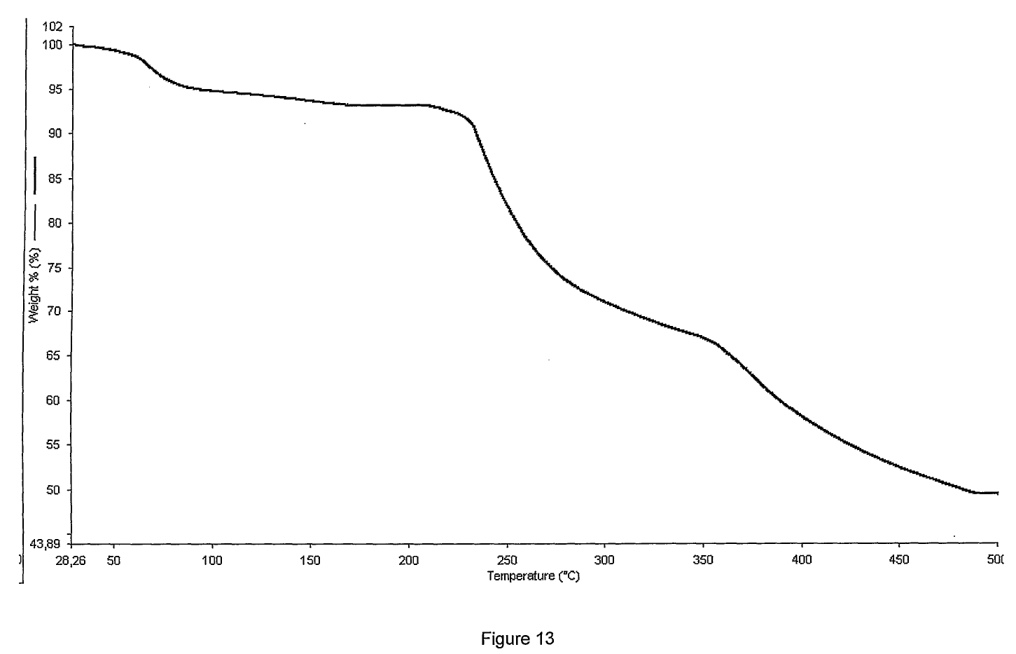

Polymorph II of valacyclovir phosphate according to the present invention is further characterised by a typical TGA thermograph, or substantially the same TGA thermograph, as shown in FIG. 13.

Polymorph II of valacyclovir phosphate according to the present invention is further characterised by a TGA weight loss of about 6.8% over the temperature range of about 30-175° C.

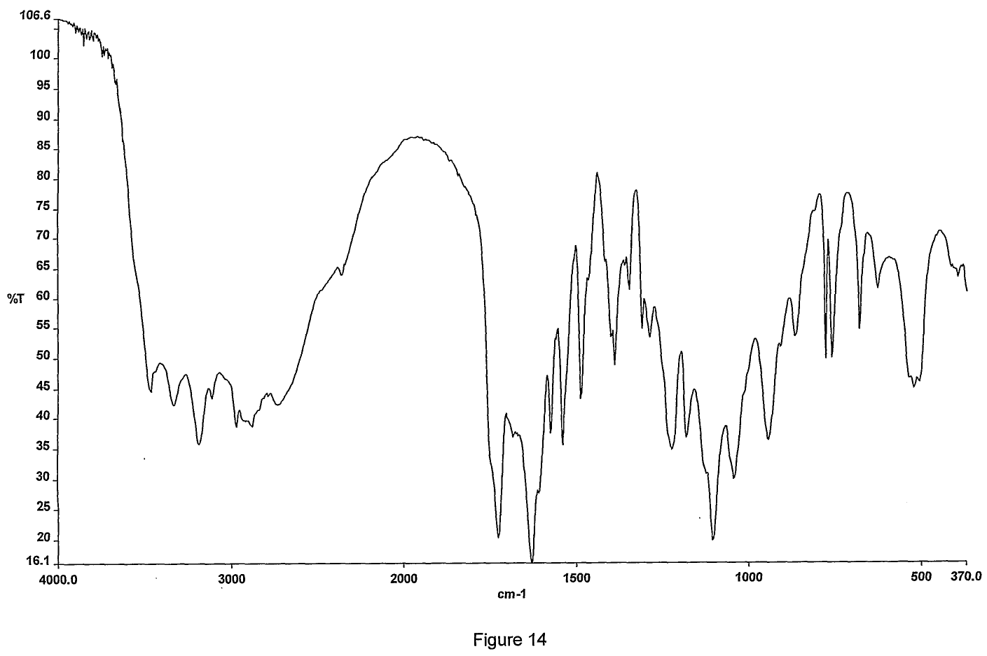

Polymorph II of valacyclovir phosphate according to the present invention is still further characterised as having an FTIR pattern, or substantially the same FTIR pattern, as shown in FIG. 14. More particularly, polymorph II of valacyclovir phosphate according to the present invention has characteristic FTIR absorbance bands at about 1727, 1630, 1541, 1288, 1225, 1184, 1046, 947, 780, 761, 681 and 524 (±14) cm⁻¹.

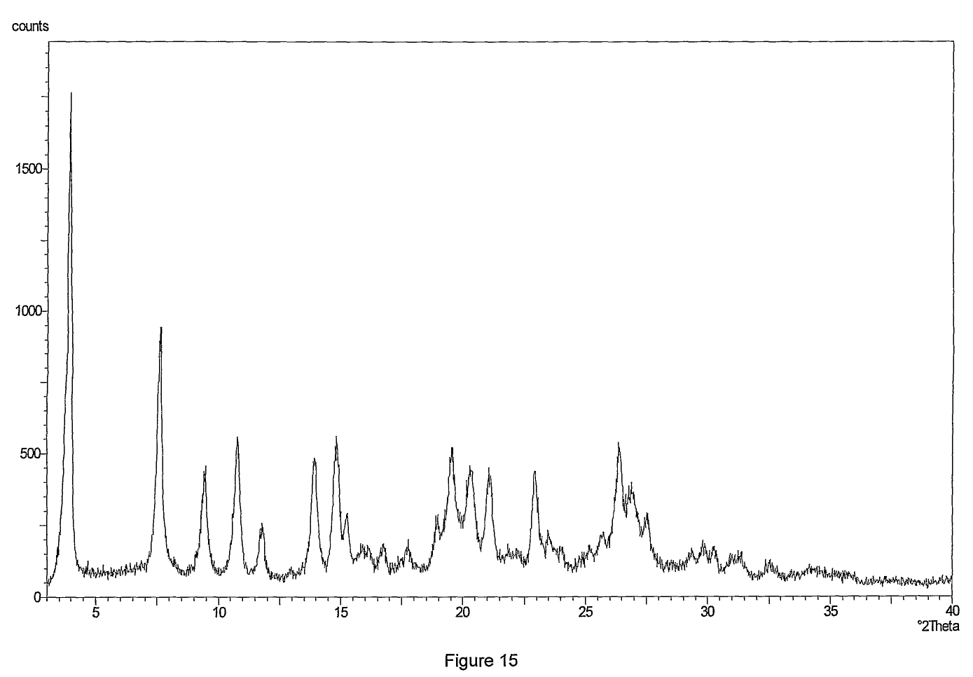

The crystalline structure of polymorph III of valacyclovir phosphate according to the present invention is characterised as having an X-ray powder diffraction pattern, or substantially the same X-ray powder diffraction pattern, as shown in FIG. 15.

Polymorph III of valacyclovir phosphate according to the present invention is further characterised as having characteristic peaks (2θ): 3.94, 7.63, 9.45, 13.96 and 14.83 (±0.2). Further peaks (2θ) associated with polymorph III of valacyclovir phosphate according to the present invention are: 10.76, 11.81, 19.51, 22.90 and 26.31 (±0.2).

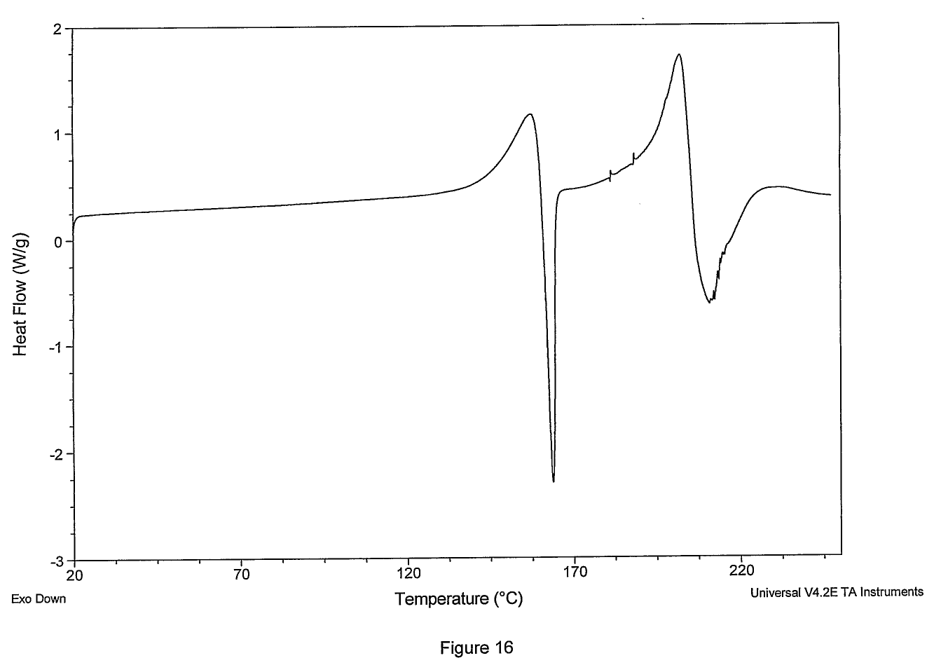

Polymorph III of valacyclovir phosphate according to the present invention is further characterised by a typical DSC thermograph as shown in FIG. 16. Polymorph III of valacyclovir phosphate has a characteristic DSC melting endotherm at about 144° C., a recrystallization exotherm at about 161° C. and a melting endotherm at about 193° C.

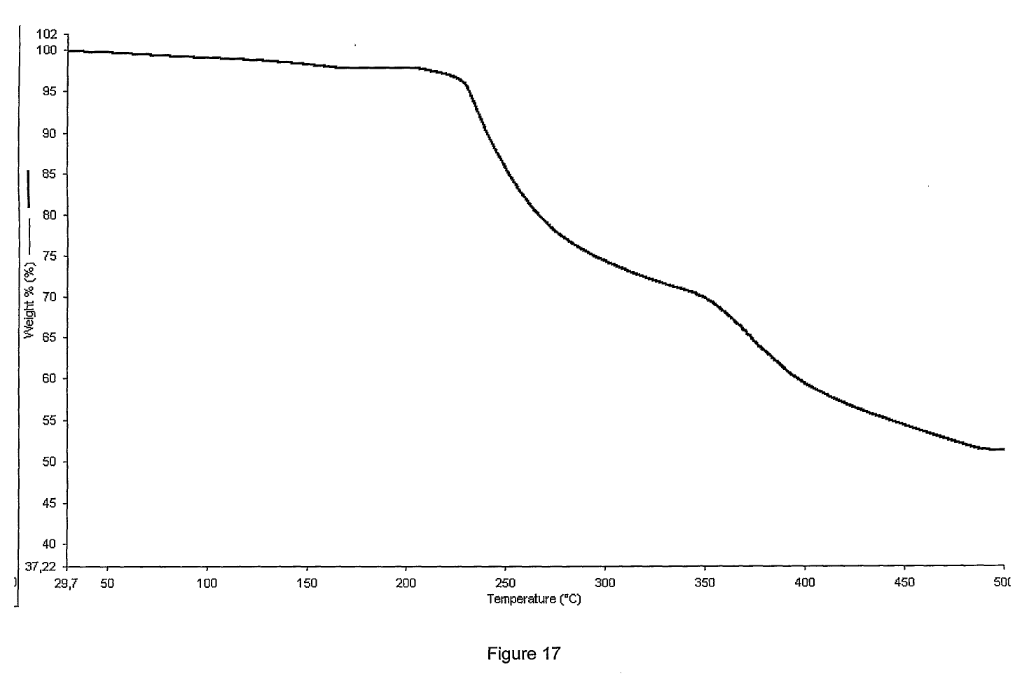

Polymorph III of valacyclovir phosphate according to the present invention is further characterised by a typical TGA thermograph, or substantially the same TGA thermograph, as shown in FIG. 17.

Polymorph III of valacyclovir phosphate according to the present invention is further characterised by a TGA weight loss of about 2.1% over the temperature range of about 30-175° C.

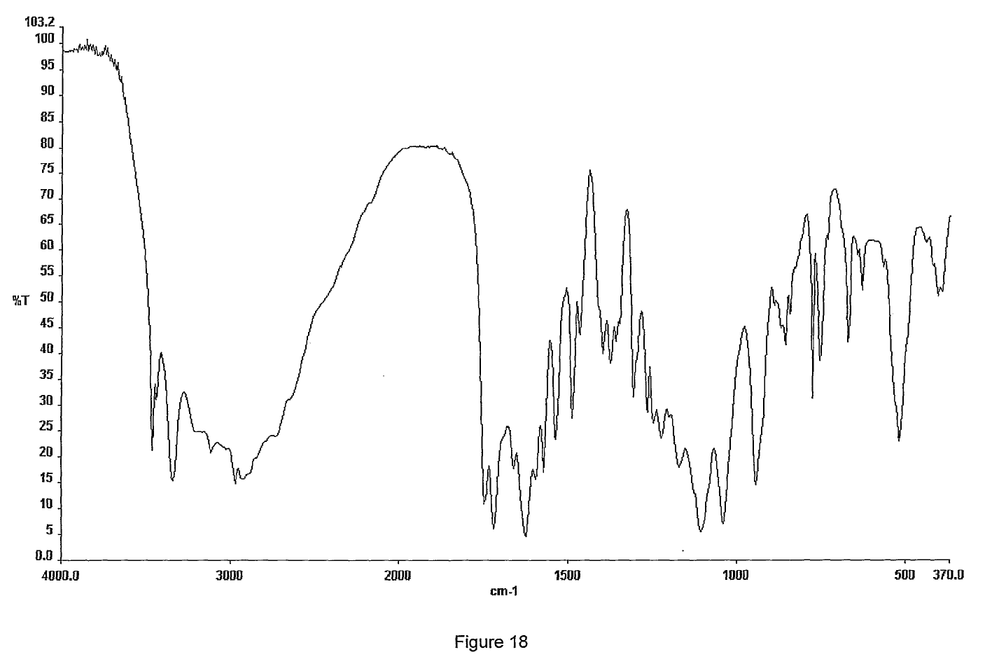

Polymorph III of valacyclovir phosphate according to the present invention is still further characterised as having an FTIR pattern, or substantially the same FTIR pattern, as shown in FIG. 18. More particularly, polymorph III of valacyclovir phosphate according to the present invention has characteristic FTIR absorbance bands at about 1749, 1720, 1661, 1376, 1267, 1042, 946, 846, 673 and 522 (±4) cm⁻¹.

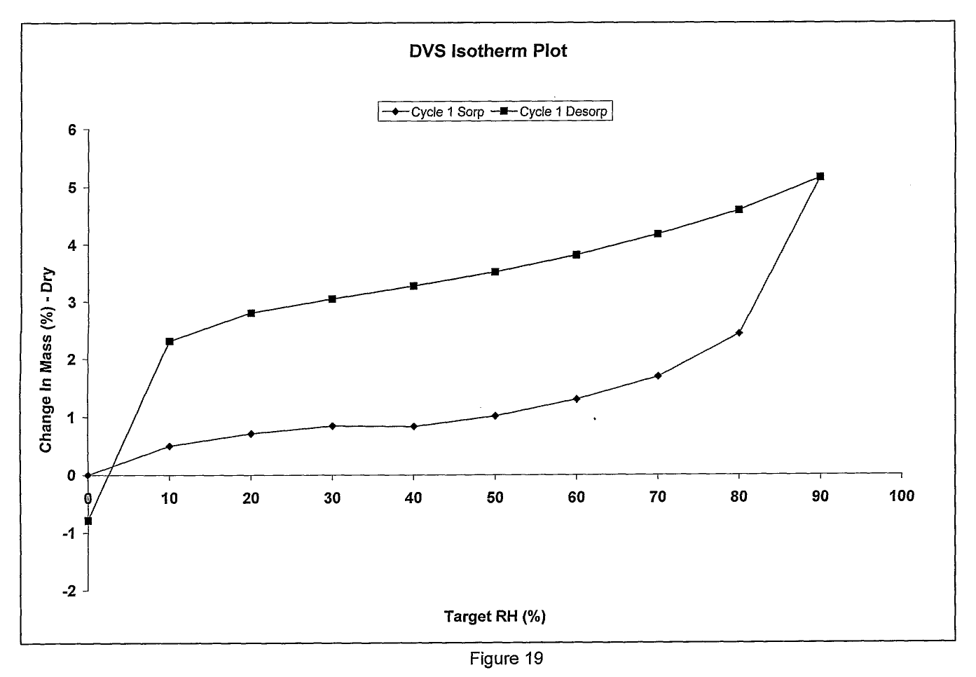

Polymorph III of valacyclovir phosphate according to the present invention can also be characterised by a typical dynamic vapour sorption (DVS) isotherm plot, or substantially the same DVS isotherm plot, as shown in FIG. 19. Polymorph III of valacyclovir phosphate according to the present invention is further characterised by a dynamic vapour sorption of about 5.2% at about 90% RH.

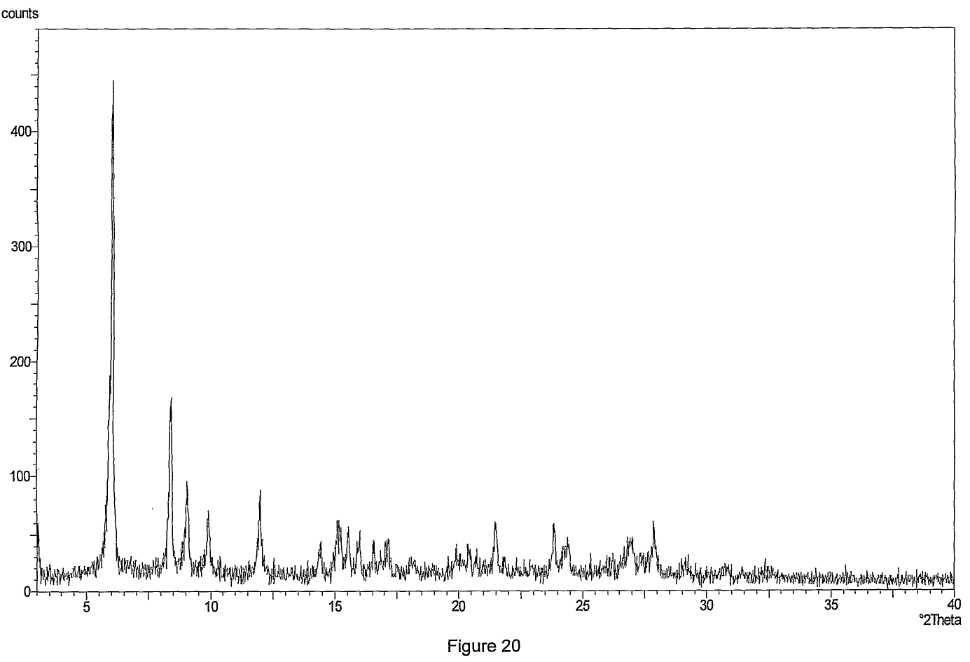

The crystalline structure of polymorph I of valacyclovir maleate according to the present invention is characterised as having an X-ray powder diffraction pattern, or substantially the same X-ray powder diffraction pattern, as shown in FIG. 20.

Polymorph I of valacyclovir maleate according to the present invention is further characterised as having characteristic peaks (2θ): 5.97, 8.96, 9.85, 11.92 and 15.48 (±0.2). Further peaks (2θ) associated with polymorph I of valacyclovir maleate according to the present invention are: 8.39, 14.33, 14.97, 21.43 and 23.81 (±0.2).

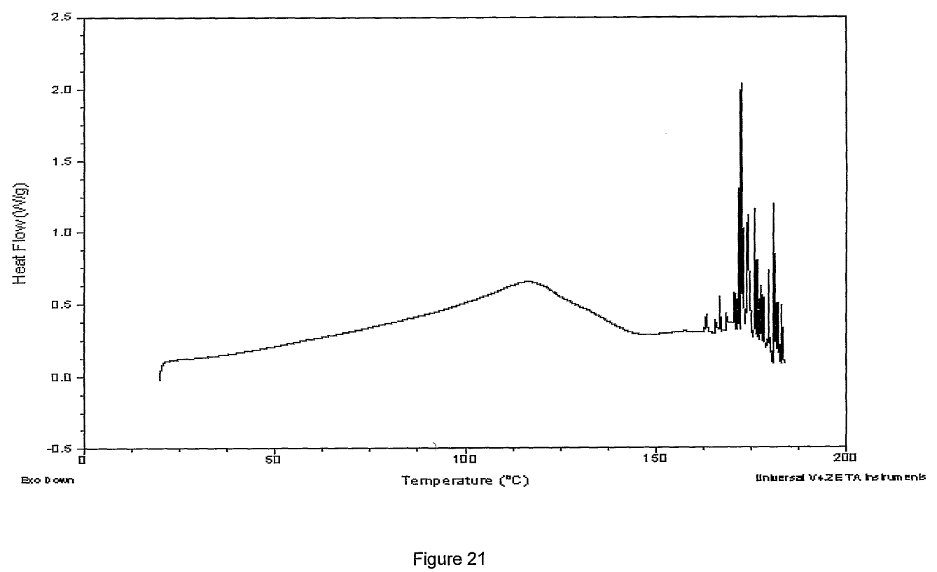

Polymorph I of valacyclovir maleate according to the present invention is further characterised by a typical DSC thermograph, originally the same DSC thermograph, as shown in FIG. 21. Polymorph I of valacyclovir maleate has a characteristic DSC endotherm representing loss of solvent and melting in the range of about 30-148° C.

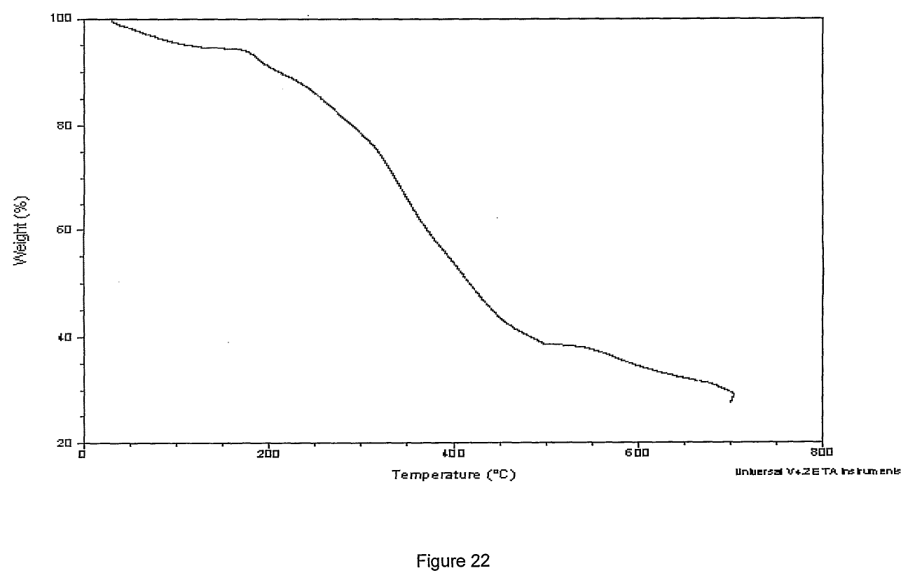

Polymorph I of valacyclovir maleate according to the present invention is further characterised by a typical TGA thermograph, or substantially the same TGA thermograph, as shown in FIG. 22.

Polymorph I of valacyclovir maleate according to the present invention is further characterised by a TGA weight loss of about 5.3% over the temperature range of about 30-150° C., which confirms that polymorph I of valacyclovir maleate as prepared according to the present invention is stable to a temperature of about 160° C.

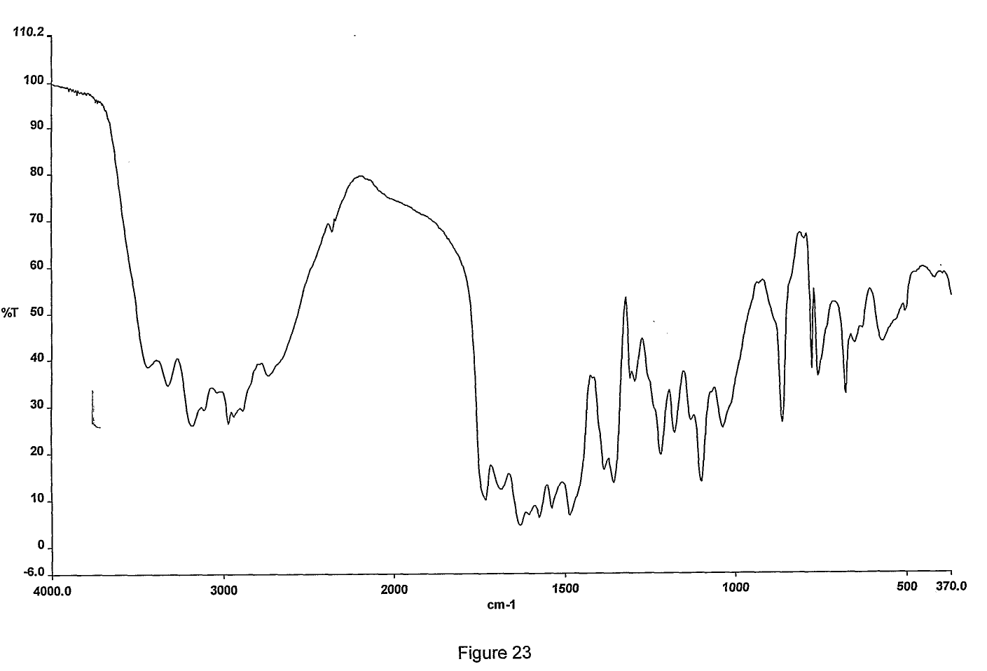

Polymorph I of valacyclovir maleate according to the present invention is still further characterised as having an FTIR pattern, or substantially the same FTIR pattern, as shown in FIG. 23. More particularly, polymorph I of valacyclovir maleate according to the present invention has characteristic FTIR absorbance bands at about 1732, 1633, 1359, 1221, 1132, 1103, 866, 681, 654 and 574 (±4) cm⁻¹.

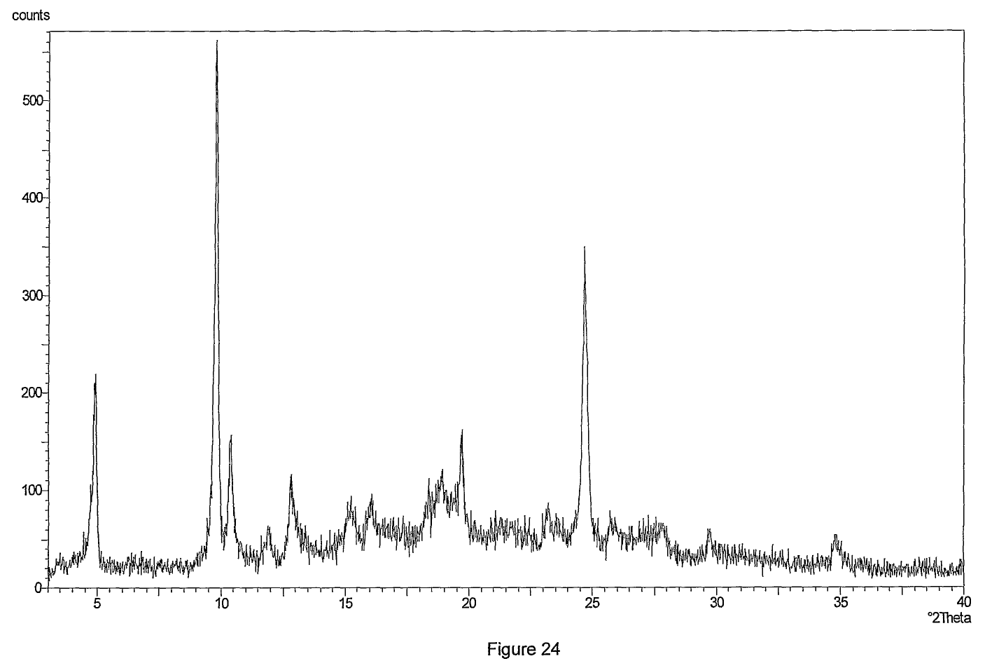

The crystalline structure of polymorph I of valacyclovir fumarate according to the present invention is characterised as having an X-ray powder diffraction pattern, or substantially the same X-ray powder diffraction pattern, as shown in FIG. 24.

Polymorph I of valacyclovir fumarate according to the present invention is further characterised as having characteristic peaks (2θ): 3.54, 7.02, 9.32, 10.57 and 11.73 (±0.2). Further peaks (2θ) associated with polymorph I of valacyclovir fumarate according to the present invention are: 14.08, 15.06, 23.58 and 26.29 (±0.2).

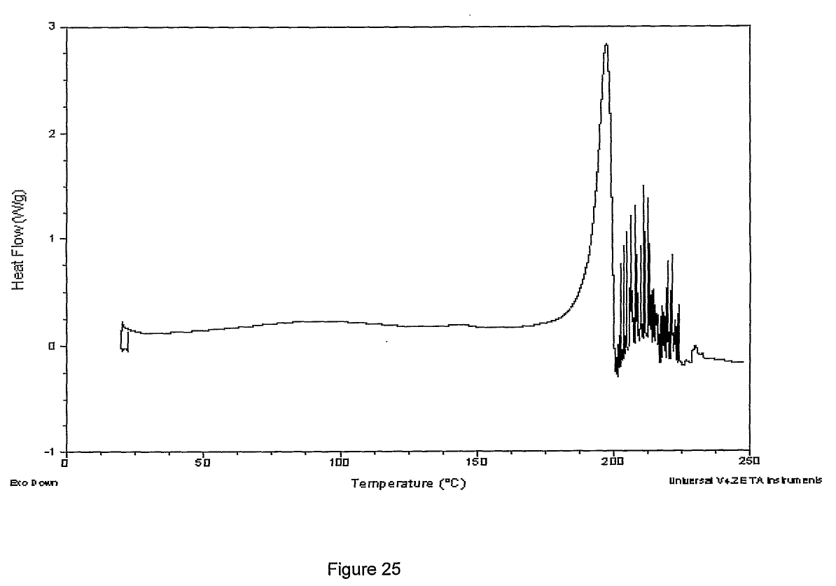

Polymorph I of valacyclovir fumarate according to the present invention is further characterised by a typical DSC thermograph, or substantially the same DSC thermograph, as shown in FIG. 25. Polymorph I of valacyclovir fumarate has a characteristic DSC melting endotherm of about 191° C.

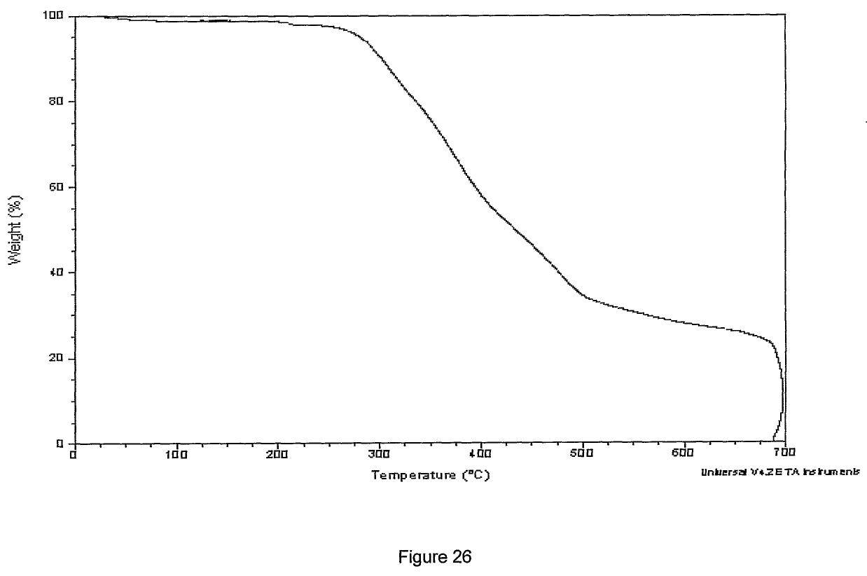

Polymorph I of valacyclovir fumarate according to the present invention is further characterised by a typical TGA thermograph, or substantially the same TGA thermograph, as shown in FIG. 26.

Polymorph I of valacyclovir fumarate according to the present invention is further characterised by a TGA weight loss of about 0.9% over the temperature range of about 30-100° C., which confirms that polymorph I of valacyclovir fumarate as prepared according to the present invention is stable to a temperature of about 200° C.

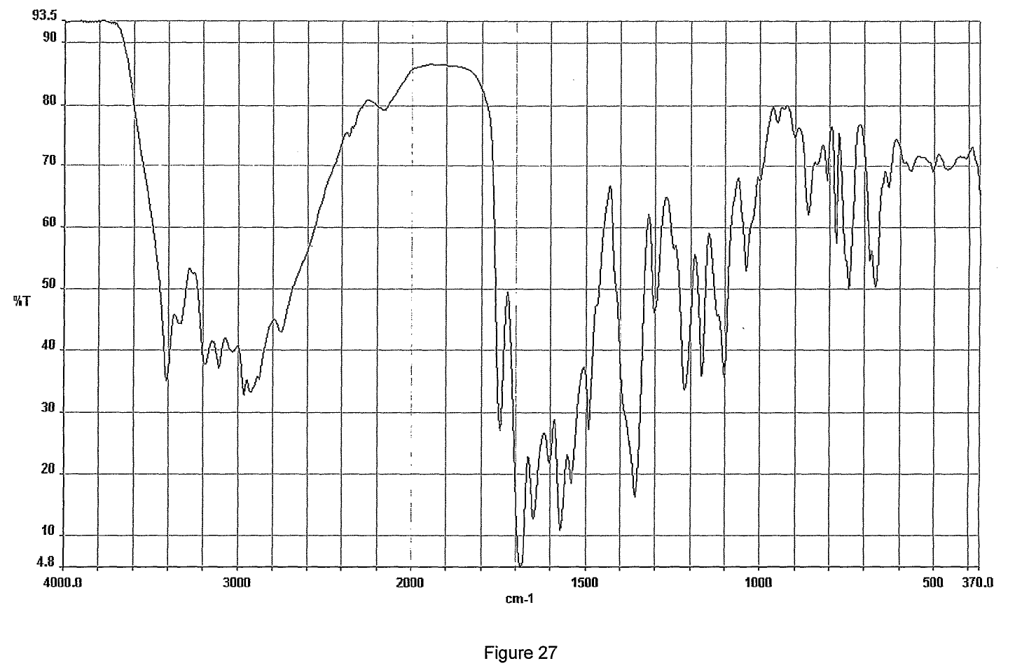

Polymorph I of valacyclovir fumarate according to the present invention is still further characterised as having an FTIR pattern, or substantially the same FTIR pattern, as shown in FIG. 27. More particularly, polymorph I of valacyclovir fumarate according to the present invention has characteristic FTIR absorbance bands at about 1748, 1687, 1573, 1360, 1218, 1168, 1104, 747 and 670 (±4) cm⁻¹.

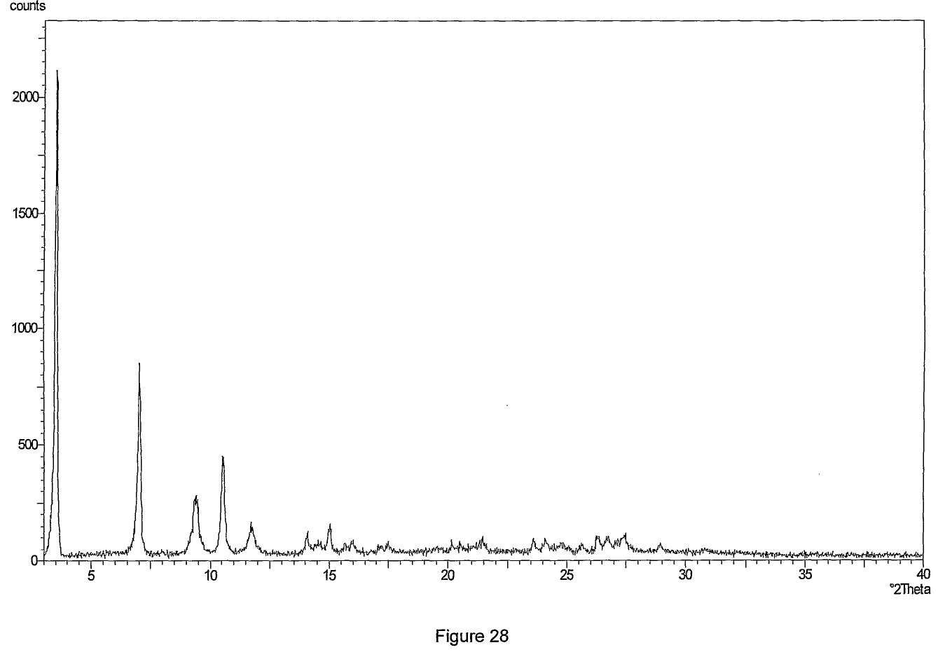

The crystalline structure of polymorph II of valacyclovir fumarate according to the present invention is characterised as having an X-ray powder diffraction pattern, or substantially the same X-ray powder diffraction pattern, as shown in FIG. 28.

Polymorph II of valacyclovir fumarate according to the present invention is further characterised as having characteristic peaks (2θ): 4.91, 9.81, 10.39, 12.80 and 24.67 (±0.2). Further peaks (2θ) associated with polymorph I of valacyclovir fumarate according to the present invention are: 11.91 and 19.69 (±0.2).

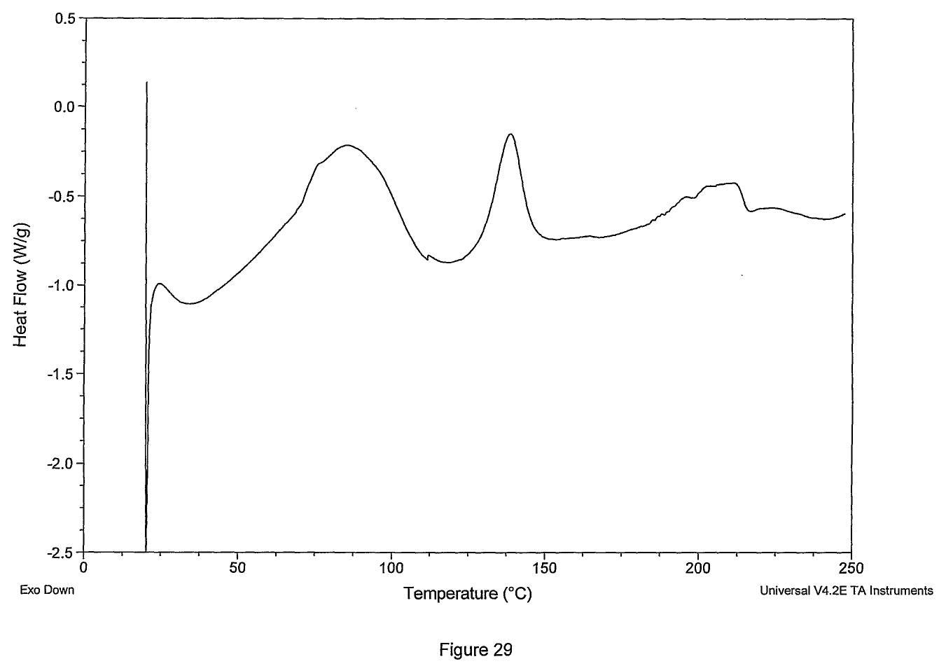

Polymorph II of valacyclovir fumarate according to the present invention is further characterised by a typical DSC thermograph, or substantially the same DSC thermograph, as shown in FIG. 29. Polymorph II of valacyclovir fumarate has a characteristic DSC endotherm representing loss of solvent in the range of about 30-120° C. and a melting endotherm at about 129° C.

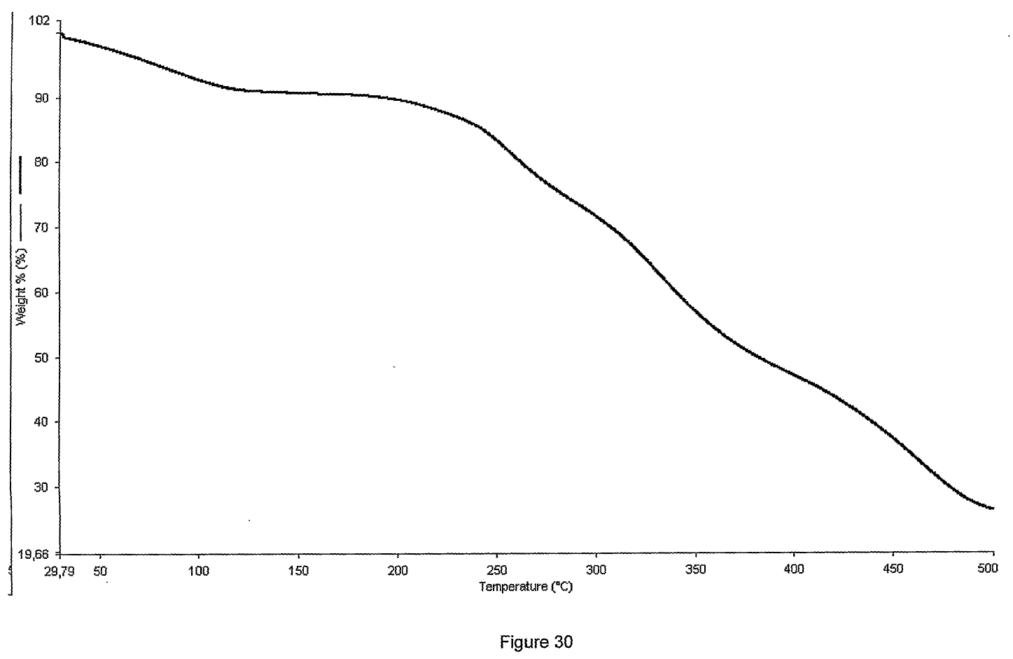

Polymorph II of valacyclovir fumarate according to the present invention is further characterised by a typical TGA thermograph, or substantially the same TGA thermograph, as shown in FIG. 30.

Polymorph II of valacyclovir fumarate according to the present invention is further characterised by a TGA weight loss of about 9.2% over the temperature range of about 30-140° C., which confirms that polymorph II of valacyclovir fumarate as prepared according to the present invention is stable to a temperature of about 150° C.

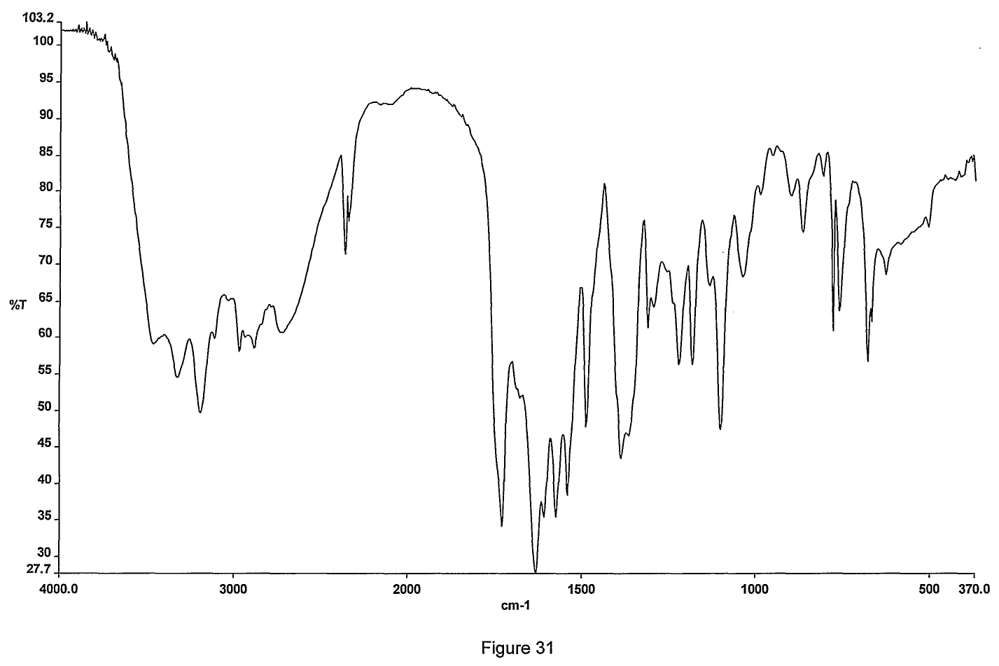

Polymorph II of valacyclovir fumarate according to the present invention is still further characterised as having an FTIR pattern, or substantially the same FTIR pattern, as shown in FIG. 31. More particularly, polymorph II of valacyclovir fumarate according to the present invention has characteristic FTIR absorbance bands at about 1729, 1632, 1574, 1488, 1388, 1102, 780, 762, 681 and 669 (±4) cm⁻¹.

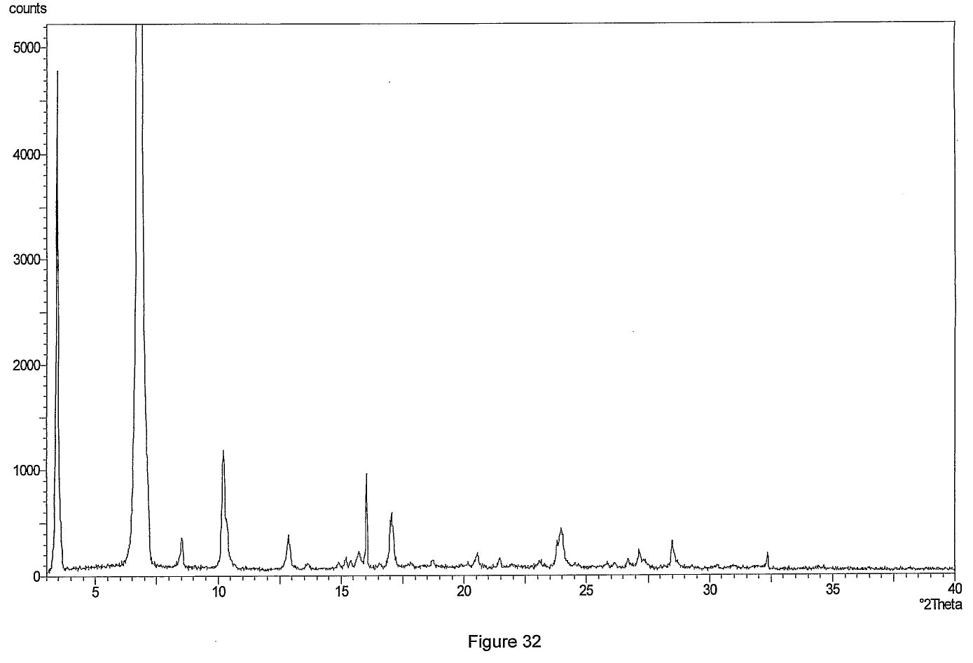

The crystalline structure of polymorph I of valacyclovir tartrate according to the present invention is characterised as having an X-ray powder diffraction pattern, or substantially the same X-ray powder diffraction pattern, as shown in FIG. 32.

Polymorph I of valacyclovir tartrate according to the present invention is further characterised as having characteristic peaks (2θ): 3.43, 6.82, 10.22, 12.85 and 16.03 (±0.2). Further peaks (2θ) associated with polymorph I of valacyclovir tartrate according to the present invention are: 8.52, 17.07, 18.72, 23.10 and 28.49 (±0.2).

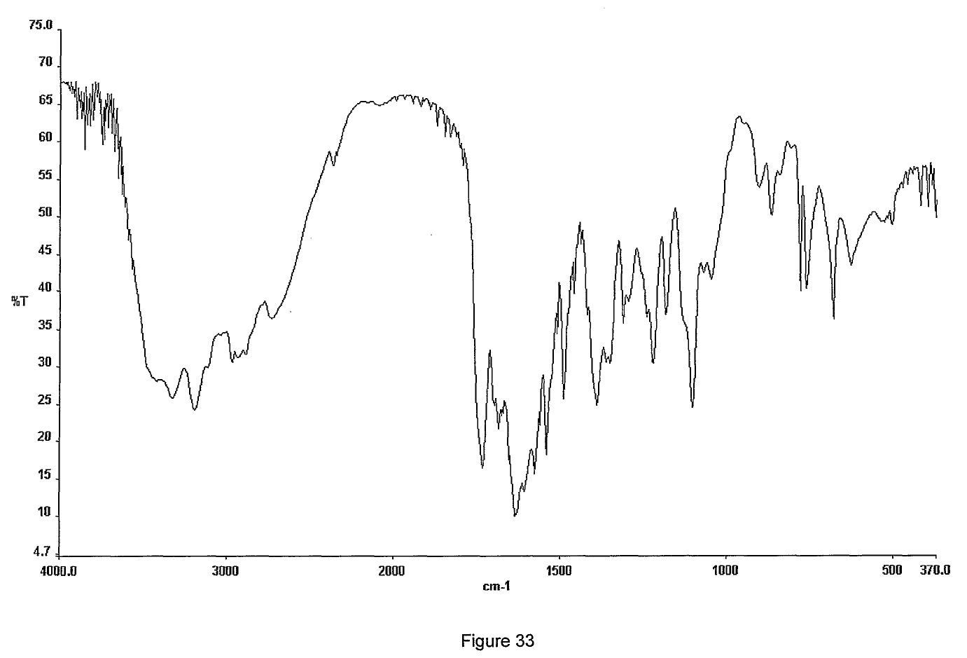

Polymorph I of valacyclovir tartrate according to the present invention is still further characterised as having an FTIR pattern, or substantially the same FTIR pattern, as shown in FIG. 33. More particularly, polymorph I of valacyclovir tartrate according to the present invention has characteristic FTIR absorbance bands at about 1733, 1635, 1541, 1489, 1389, 1350, 1221, 1103, 780, 762 and 682 (±4) cm⁻¹.

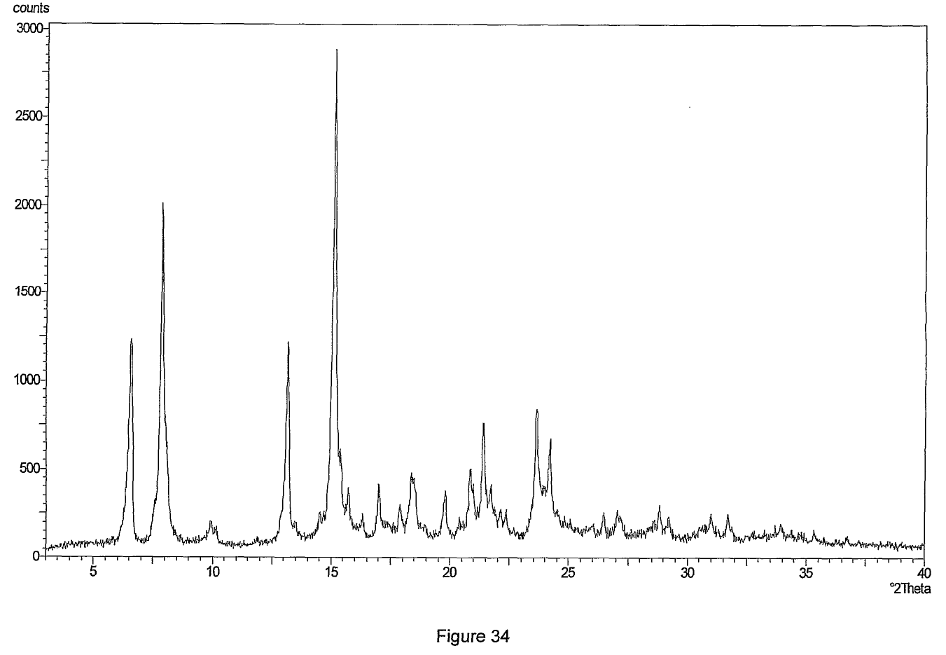

The crystalline structure of polymorph I of valacyclovir citrate according to the present invention is characterized as having an X-ray powder diffraction pattern, or substantially the same X-ray powder diffraction pattern, as shown in FIG. 34.

Polymorph I of valacyclovir citrate according to the present invention is further characterised as having characteristic peaks (2θ): 6.59, 7.86, 13.18, 15.13 and 17.00 (±0.2). Further peaks (2θ) associated with polymorph I of valacyclovir citrate according to the present invention are: 15.74, 18.35, 18.98, 19.82, 21.39 and 23.64 (±0.2).

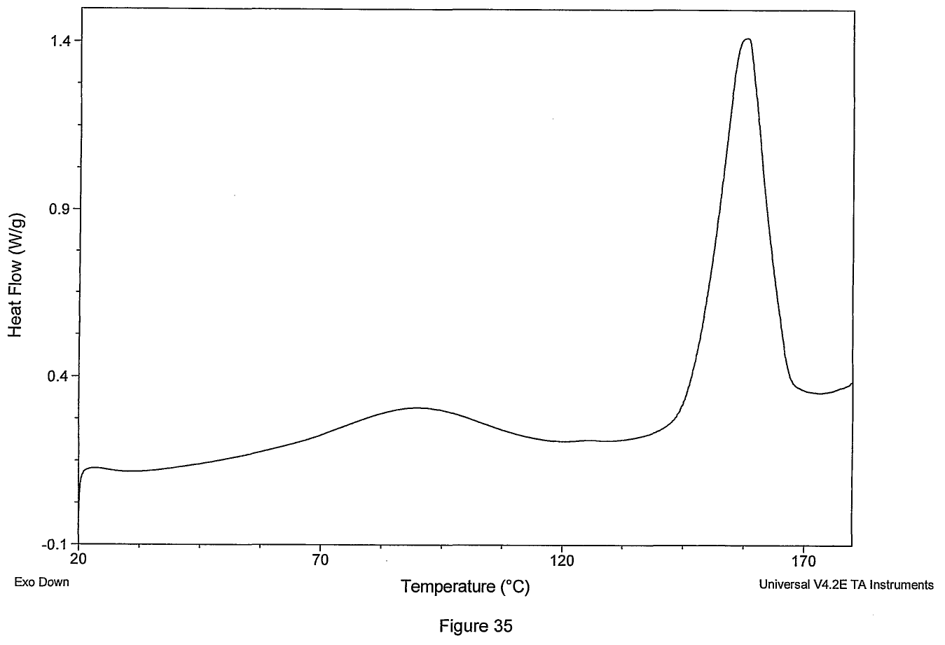

Polymorph I of valacyclovir citrate according to the present invention is further characterised by a typical DSC thermograph, or substantially the same DSC thermograph, as shown in FIG. 35. Polymorph I of valacyclovir citrate has a characteristic DSC endotherm representing loss of solvent in the range of 30-120° C. and melting endotherm at about 147° C.

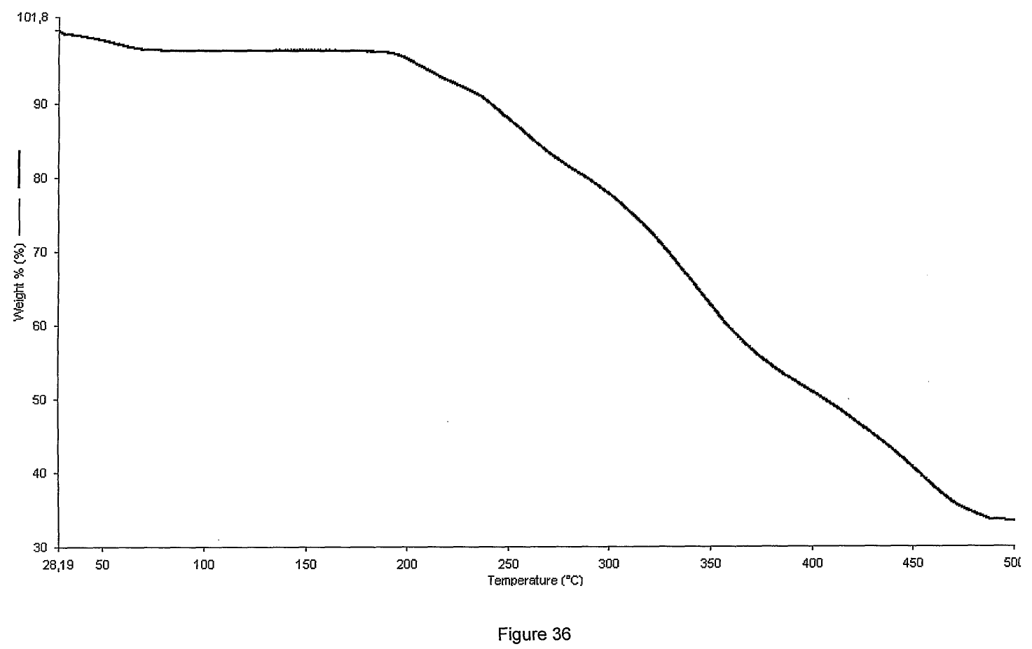

Polymorph I of valacyclovir citrate according to the present invention is further characterised by a typical TGA thermograph, or substantially the same TGA thermograph, as shown in FIG. 36.

Polymorph I of valacyclovir citrate according to the present invention is further characterised by a TGA weight loss of about 2.6% over the temperature range of about 30-80° C., which confirms that polymorph I of valacyclovir citrate as prepared according to the present invention is stable to a temperature of about 180° C.

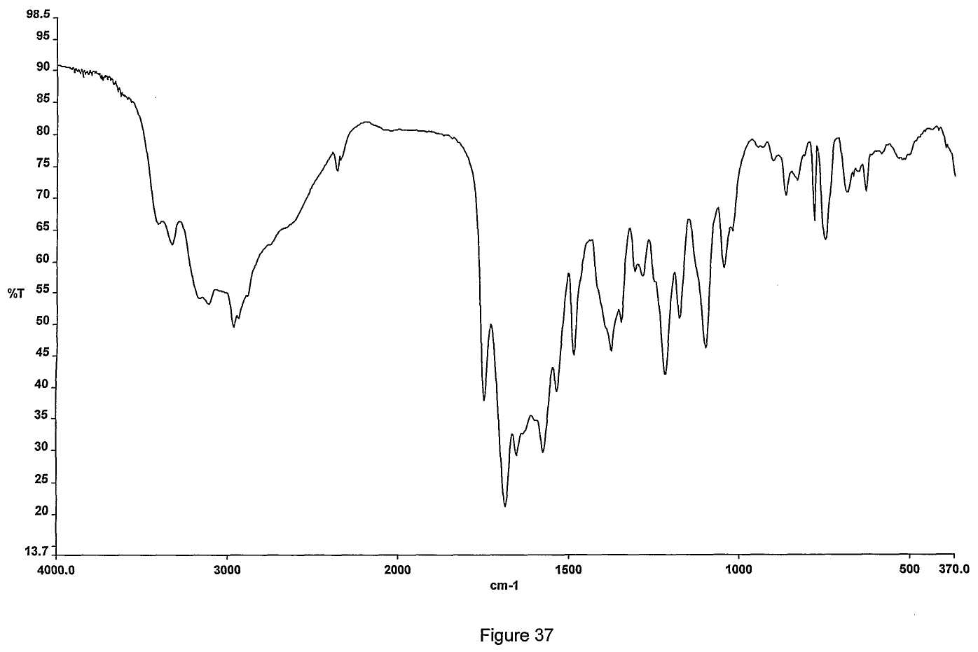

Polymorph I of valacyclovir citrate according to the present invention is still further characterised as having an FTIR pattern, or substantially the same FTIR pattern, as shown in FIG. 37. More particularly, polymorph I of valacyclovir citrate according to the present invention has characteristic FTIR absorbance bands at about 1749, 1687, 1576, 1487, 1377, 1219, 1101, 783 and 750 (±4) cm⁻¹.

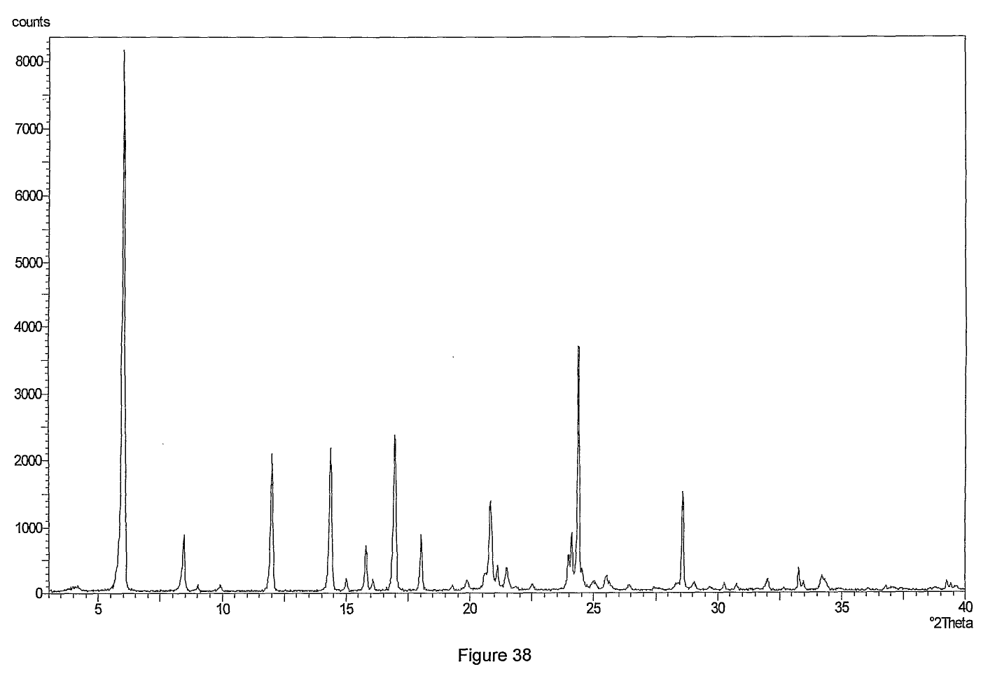

The crystalline structure of polymorph I of valacyclovir base according to the present invention is characterised as having an X-ray powder diffraction pattern, or substantially the same X-ray powder diffraction pattern, as shown in FIG. 38.

Polymorph I of valacyclovir base according to the present invention is further characterised as having characteristic peaks (2θ): 6.03, 12.01, 14.38, 16.98 and 18.03 (±0.2). Further peaks (2θ) associated with polymorph I of valacyclovir base according to the present invention are: 8.47, 9.93, 15.02, 15.80 and 24.37 (±0.2).

The crystalline structure of polymorph I of valacyclovir base according to the present invention is characterised by monoclinic space group P12₁1 displaying unit cell parameters comprising crystal axis lengths of a=4.66±0.01 Å, b=11.22±0.01 Å, c=29.53±0.01 Å and angles between the crystal axes of α=90.00°±0.01, β=90.46°±0.01 and γ=90.00±0.01°. The crystalline structure of polymorph I of valacyclovir base is further characterised by the following properties:

| Empirical formula | C₁₃H₂₀N₆O₄ | |
|---|---|---|
| Formula weight | 324.33 | |
| Volume | 1545.29 | Å³ |
| Z, calculated density | 2, 1.39 | g/cm³ |
| Wavelength | 1.54184 | Å |

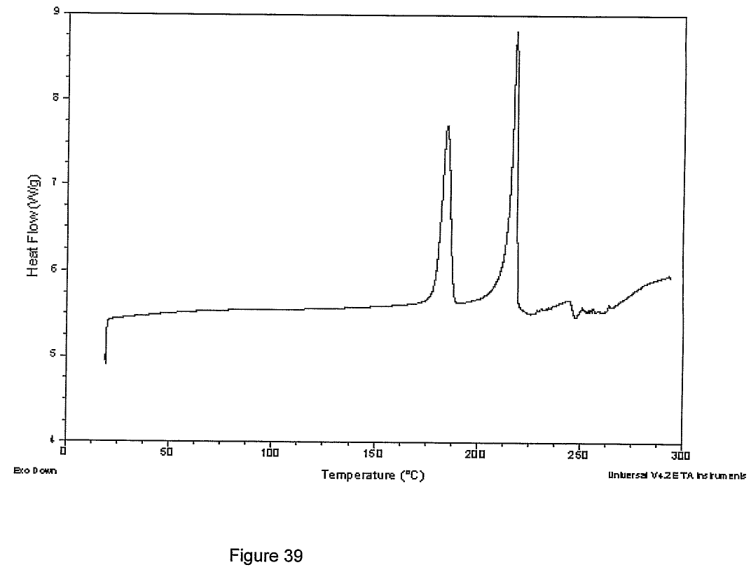

Polymorph I of valacyclovir base according to the present invention is further characterised by a typical DSC thermograph, or substantially the same DSC thermograph, as shown in FIG. 39. Polymorph I of valacyclovir hemicitrate has a characteristic DSC melting endotherm at about 180° C. and about 214° C.

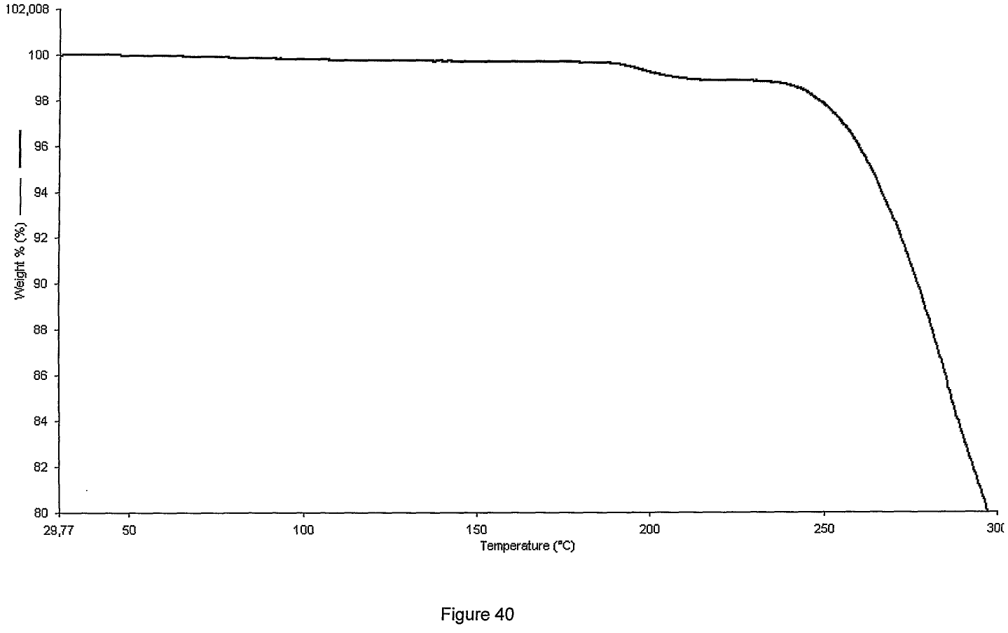

Polymorph I of valacyclovir base according to the present invention is further characterised by a typical TGA thermograph, or substantially the same TGA thermograph, as shown in FIG. 40. Polymorph I of valacyclovir base according to the present invention is further characterised by no TGA weight loss over the temperature range of about 200° C., which confirms that polymorph I of valacyclovir base as prepared according to the present invention is stable to a temperature of about 200° C.

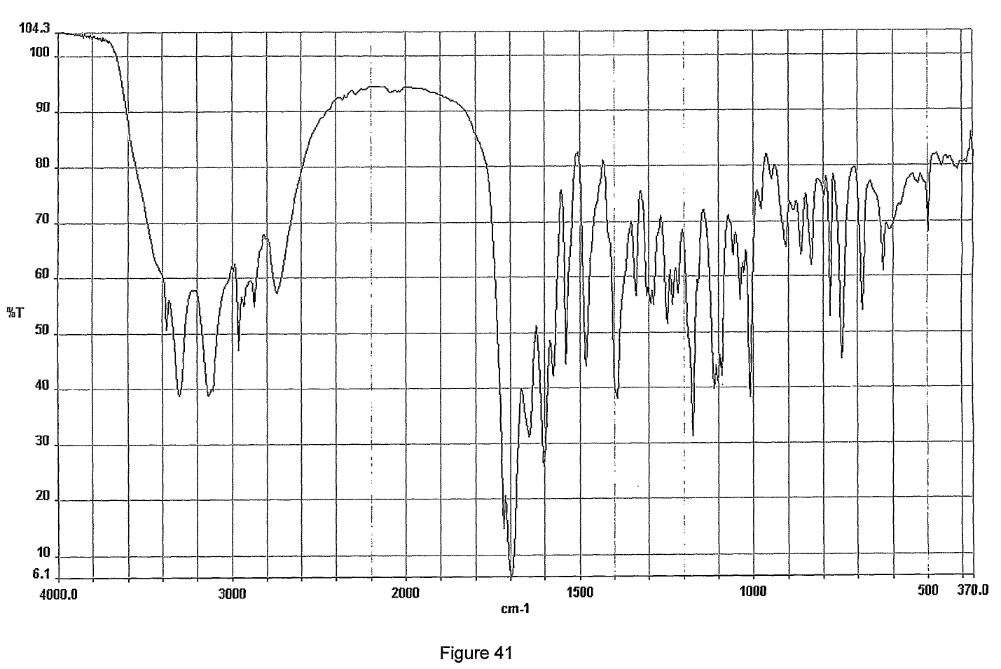

Polymorph I of valacyclovir base according to the present invention is still further characterised as having an FTIR pattern, or substantially the same FTIR pattern, as shown in FIG. 41.

More particularly, polymorph I of valacyclovir base according to the present invention has characteristic FTIR absorbance bands at about 1720, 1699, 1605, 1484, 1394, 1176, 1012, 782, 747 and 668 cm⁻¹ (±4 cm⁻¹).

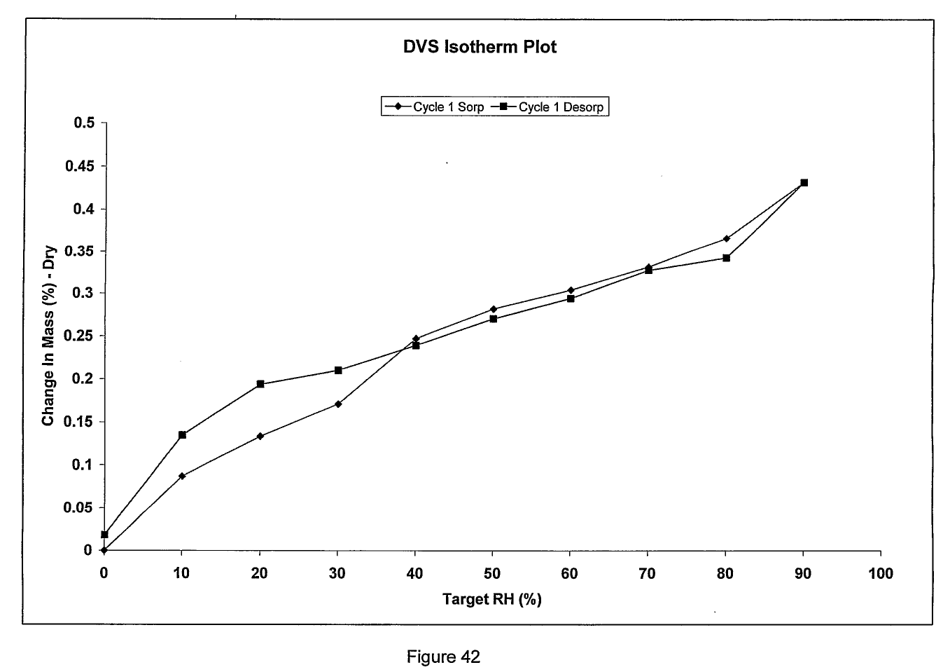

Polymorph I of valacyclovir base according to the present invention can also be characterised by a typical dynamic vapour sorption (DVS) isotherm plot, or substantially the same DVS isotherm plot, as shown in FIG. 42.

Polymorph I of valacyclovir base according to the present invention is further characterised by a dynamic vapour sorption of about 0.4% at about 90% RH.

There is also provided by the present invention processes for preparing pharmaceutically acceptable salts of valacyclovir substantially as hereinbefore described and also the polymorphic forms thereof as described herein.

According to the present invention there is further provided a process of preparing a pharmaceutically acceptable salt of valacyclovir substantially as hereinbefore described, which process comprises treating valacyclovir free base with a pharmaceutically acceptable acid selected from the group consisting of methanesulphonic acid, phosphoric acid, maleic acid, fumaric acid, tartaric acid and citric acid.

Typically, the process can comprise suspending valacyclovir base in a suitable medium and adding a pharmaceutically acceptable acid dissolved in a suitable solvent. Suitable media include ethanol and/or methanol. Suitable solvents for the pharmaceutically acceptable acid include ethanol and/or methanol.

When mixing valacyclovir salt or free base in a medium to form a solution or a suspension, warming of the mixture can be necessary to completely dissolve the starting material. If warming does not clarify the mixture, the mixture can be diluted or filtered.

Depending upon the equipment used and the concentration and temperature of the solution, the filtration apparatus may need to be preheated to avoid premature crystallization.

The conditions can also be changed to induce precipitation. In one embodiment the solubility of the solvent can be reduced, for example, by cooling the solvent.

In one embodiment, an anti-solvent is added to a solution to decrease its solubility for a particular compound, thus resulting in precipitation.

Another manner to accelerate crystallization is by seeding with a crystal of the product or scratching the inner surface of the crystallization vessel with a glass rod.

Other times, crystallization can occur spontaneously without any inducement. All that is necessary to be within the scope of the claims is to form a precipitate or crystal.

The precipitate or crystal may undergo further steps such as drying, filtering, washing and recrystallization.

There is also provided a process of polymorph interconversion, which process comprises converting a first polymorphic form of a pharmaceutically acceptable salt of valacyclovir as prepared by the above process to a further polymorphic form of the pharmaceutically acceptable valacyclovir salt. Typically the interconversion can comprise dissolving (often under reflux conditions) a first polymorphic form in a suitable solvent, such as for example water, a mixture of water and one or more alcohols, a mixture of water and acetonitrile or a mixture of water and benzonitrile and allowing crystals of the further polymorphic form to form. Examples of suitable alcohols include methanol, ethanol, 1-propanol, 2-propanol and benzylalcohol. A specific example of this means of interconversion is the preparation of valacyclovir fumarate form II from valacyclovir fumarate form I.

Alternatively, a particular form can be dried, optionally in a vacuum, over a prolonged period of time to yield a different polymorphic form. A specific example of this means of interconversion is the preparation of valacyclovir phosphate form III from valacyclovir phosphate form II.

Alternatively, a particular polymorphic form can be exposed to an elevated relative humidity to yield a different polymorphic form, which under such conditions is typically hydrated. A specific example of this means of interconversion is the preparation of valacyclovir phosphate form II from valacyclovir phosphate form I.

Valacyclovir salts and polymorphic forms as provided by the present invention are L-valyl ester prodrugs of acyclovir, being rapidly and almost completely converted in vivo by first-pass metabolism to acyclovir. Acyclovir is an acyclic guanine nucleoside analogue which has been found to have potent anti-viral activity and is widely used in the treatment and prophylaxis of viral infections, particularly infections caused by the herpes group of viruses. Valacyclovir salts and polymorphs as provided by the present invention are thus useful in the treatment and prevention of viral infections, particularly infections caused by the herpes group of viruses.

The present invention further provides, therefore, pharmaceutical compositions comprising a therapeutically effective dose of a valacyclovir salt or polymorphic form according to the invention, together with a pharmaceutically acceptable carrier, diluent or excipient therefor. Excipients are chosen according to the pharmaceutical form and the desired mode of administration.

As used herein, the term “therapeutically effective amount” means an amount of a valacyclovir salt or polymorphic form according to the invention, which is capable of preventing, ameliorating or eliminating a disease state for which administration of a compound having anti-viral activity is indicated.

By “pharmaceutically acceptable” it is meant that the carrier, diluent or excipient is compatible with a valacyclovir salt or polymorphic form according to the invention, and not deleterious to a recipient thereof.

In the pharmaceutical compositions of the present invention for oral, sublingual, subcutaneous, intramuscular, intravenous, topical, intratracheal, intranasal, transdermal or rectal administration, a valacyclovir salt or polymorphic form according to the present invention is administered to animals and humans in unit forms of administration, mixed with conventional pharmaceutical carriers, for the prophylaxis or treatment of the above disorders or diseases. The appropriate unit forms of administration include forms for oral administration, such as tablets, gelatin capsules, powders, granules and solutions or suspensions to be taken orally, forms for sublingual, buccal, intratracheal or intranasal administration, forms for subcutaneous, intramuscular or intravenous administration and forms for rectal administration. For topical application, a valacyclovir salt or polymorphic form according to the present invention can be used in creams, ointments or lotions. Oral administration is preferred.

To achieve the desired prophylactic or therapeutic effect, the dose of a valacyclovir salt or polymorphic form according to the present invention can vary between 0.01 and 50 mg per kg of body weight per day. Each unit dose can contain from 0.1 to 1000 mg, preferably 1 to 500 mg, of a valacyclovir salt or polymorphic form according to the present invention in combination with a pharmaceutical carrier. This unit dose can be administered 1 to 5 times a day so as to administer a daily dosage of 0.5 to 5000 mg, preferably 1 to 2500 mg.

When a solid composition in the form of tablets is prepared, a valacyclovir salt or polymorphic form according to the present invention is mixed with a pharmaceutical vehicle such as gelatin, starch, lactose, magnesium stearate, talc, gum arabic or the like. The tablets can be coated with sucrose, a cellulose derivative or other appropriate substances, or else they can be treated so as to have a prolonged or delayed activity and so as to release a predetermined amount of active principle continuously.

A preparation in the form of gelatin capsules can be obtained by mixing a valacyclovir salt or polymorphic form according to the present invention with a diluent and pouring the resulting mixture into soft or hard gelatin capsules.

A preparation in the form of a syrup or elixir or for administration in the form of drops can contain a valacyclovir salt or polymorphic form according to the present invention typically in conjunction with a sweetener, which is preferably calorie-free, optionally antiseptics such as methylparaben and propylparaben, as well as a flavoring and an appropriate color.

Water-dispersible granules or powders can contain a valacyclovir salt or polymorphic form according to the present invention mixed with dispersants or wetting agents, or suspending agents such as polyvinylpyrrolidone, as well as with sweeteners or taste correctors.

Rectal administration is effected using suppositories prepared with binders which melt at the rectal temperature, for example polyethylene glycols.

Parenteral administration is effected using aqueous suspensions, isotonic saline solutions or sterile and injectable solutions which contain pharmacologically compatible dispersants and/or wetting agents, for example propylene glycol or butylene glycol.

A valacyclovir salt or polymorphic form according to the present invention can also be formulated as microcapsules, with one or more carriers or additives if appropriate.

There is also provided by the present invention a valacyclovir salt or polymorphic form substantially as hereinbefore described for use in therapy.

The present invention further provides a valacyclovir salt or polymorphic form substantially as hereinbefore described, for use in the manufacture of a medicament for the treatment of a disease state prevented, ameliorated or eliminated by the administration of a compound having anti-viral activity. More specifically, the present invention provides a valacyclovir salt or polymorphic form substantially as hereinbefore described, for use in the manufacture of a medicament for the treatment or prevention of viral infections, particularly those caused by the herpes group of viruses.

The present invention also provides a method of treating a disease state prevented, ameliorated or eliminated by the administration of a compound having anti-viral activity to a patient in need of such treatment, which method comprises administering to the patient a therapeutically effective amount of a valacyclovir salt or polymorphic form substantially as hereinbefore described. More specifically, the present invention provides a method of treating or preventing viral infections, particularly those caused by the herpes group of viruses.

There is also provided by the present invention a valacyclovir salt or polymorphic substantially as hereinbefore described, for use in the manufacture of a medicament for the treatment of a disease state prevented, ameliorated or eliminated by the administration of a compound having anti-viral activity, wherein said valacyclovir salt or polymorphic form according to the invention, provides an enhanced therapeutic effect compared to the therapeutic effect provided by valacyclovir hydrochloride. The present invention also provides a corresponding method of treatment, which comprises administering to a patient a therapeutically effective amount of a valacyclovir salt or polymorphic form substantially as hereinbefore described, so that the administered valacyclovir salt or polymorphic form according to the present invention, provides an enhanced therapeutic effect to the patient, compared to the therapeutic effect provided by corresponding administration of valacyclovir hydrochloride.

The present invention can be further illustrated by the following Figures and non-limiting Examples.

With reference to the Figures, these are as follows:

FIG. 1: X-ray powder diffraction pattern of polymorph I of valacyclovir mesylate according to the present invention obtained by using a Philips X'Pert PRO with CuKα radiation in 2θ=3-40° range.

FIG. 2: Typical DSC thermograph of polymorph I of valacyclovir mesylate obtained by using a DSC Pyris 1 manufactured by Perkin-Elmer. The experiment was done under a flow of nitrogen (35 ml/min) and heating rate was 10° C./min. A standard sample pan was used.

FIG. 3: Typical TGA thermograph of polymorph I of valacyclovir mesylate obtained by using thermogravimetric analysis (TGA) using TGA 7 manufactured by Perkin-Elmer. The experiments were done under flow of nitrogen (35 ml/min) and heating rate was 10° C./min.

FIG. 4: FTIR pattern of polymorph I of valacyclovir mesylate obtained by using a KBr pellet and Spectrum GX manufactured by Perkin-Elmer. Resolution was 4 cm⁻¹.

FIG. 5: Typical DVS isotherm plot of polymorph I of valacyclovir mesylate

FIG. 6: X-ray powder diffraction pattern of polymorph I of valacyclovir phosphate according to the present invention obtained by using a Philips X'Pert PRO with CuKα radiation in 2θ=3-40° range.

FIG. 7: Typical DSC thermograph of polymorph I of valacyclovir phosphate obtained by using a DSC Pyris 1 manufactured by Perkin-Elmer. The experiment was done under a flow of nitrogen (35 ml/min) and heating rate was 10° C./min. A standard sample pan was used.

FIG. 8: Typical TGA thermograph of polymorph I of valacyclovir phosphate obtained by using thermogravimetric analysis (TGA) using TGA 7 manufactured by PerkinElmer. The experiments were done under flow of nitrogen (35 ml/min) and heating rate was 10° C./min.

FIG. 9: FTIR pattern of polymorph I of valacyclovir phosphate obtained by using a KBr pellet and Spectrum GX manufactured by Perkin-Elmer. Resolution was 4 cm⁻¹.

FIG. 10: Typical DVS isotherm plot of polymorph I of valacyclovir phosphate

FIG. 11: X-ray powder diffraction pattern of polymorph II of valacyclovir phosphate according to the present invention obtained by using a Philips X'Pert PRO with CuKα radiation in 2θ=3-40° range

FIG. 12: Typical DSC thermograph of polymorph II of valacyclovir phosphate obtained by using a DSC Pyris 1 manufactured by Perkin-Elmer. The experiment was done under a flow of nitrogen (35 ml/min) and heating rate was 10° C./min. A standard sample pan was used.

FIG. 13: Typical TGA thermograph of polymorph II of valacyclovir phosphate obtained by using thermogravimetric analysis (TGA) using TGA 7 manufactured by PerkinElmer. The experiments were done under flow of nitrogen (35 ml/min) and heating rate was 10° C./min.

FIG. 14: FTIR pattern of polymorph II of valacyclovir phosphate obtained by using a KBr pellet and Spectrum GX manufactured by Perkin-Elmer. Resolution was 4 cm⁻¹.

FIG. 15: X-ray powder diffraction pattern of polymorph III of valacyclovir phosphate according to the present invention obtained by using a Philips X'Pert PRO with CuKα radiation in 2θ=3-40° range.

FIG. 16: Typical DSC thermograph of polymorph III of valacyclovir phosphate obtained by using a DSC Pyris 1 manufactured by Perkin-Elmer. The experiment was done under a flow of nitrogen (35 ml/min) and heating rate was 10° C./min. A standard sample pan was used.

FIG. 17: Typical TGA thermograph of polymorph III of valacyclovir phosphate obtained by using thermogravimetric analysis (TGA) using TGA 7 manufactured by PerkinElmer. The experiments were done under flow of nitrogen (35 ml/min) and heating rate was 10° C./min.

FIG. 18: FTIR pattern of polymorph III of valacyclovir phosphate obtained by using a KBr pellet and Spectrum GX manufactured by Perkin-Elmer. Resolution was 4 cm⁻¹.

FIG. 19: Typical DVS isotherm plot of polymorph III of valacyclovir phosphate

FIG. 20: X-ray powder diffraction pattern of polymorph I of valacyclovir maleate according to the present invention obtained by using a Philips X'Pert PRO with CuKα radiation in 2θ=3-40° range.

FIG. 21: Typical DSC thermograph of polymorph I of valacyclovir maleate obtained by using a DSC Pyris 1 manufactured by Perkin-Elmer. The experiment was done under a flow of nitrogen (35 ml/min) and heating rate was 10° C./min. A standard sample pan was used.

FIG. 22: Typical TGA thermograph of polymorph I of valacyclovir maleate obtained by using thermogravimetric analysis (TGA) using TGA 7 manufactured by Perkin-Elmer. The experiments were done under flow of nitrogen (35 ml/min) and heating rate was 10° C./min.

FIG. 23: FTIR pattern of polymorph I of valacyclovir maleate obtained by using a KBr pellet and Spectrum GX manufactured by Perkin-Elmer. Resolution was 4 cm⁻¹.

FIG. 24: X-ray powder diffraction pattern of polymorph I of valacyclovir fumarate according to the present invention obtained by using a Philips X'Pert PRO with CuKα radiation in 2θ=3-40° range.

FIG. 25: Typical DSC thermograph of polymorph I of valacyclovir fumarate obtained by using a DSC Pyris 1 manufactured by Perkin-Elmer. The experiment was done under a flow of nitrogen (35 ml/min) and heating rate was 10° C./min. A standard sample pan was used.

FIG. 26: Typical TGA thermograph of polymorph I of valacyclovir fumarate obtained by using thermogravimetric analysis (TGA) using TGA 7 manufactured by Perkin-Elmer. The experiments were done under flow of nitrogen (35 ml/min) and heating rate was 10° C./min.

FIG. 27: FTIR pattern of polymorph I of valacyclovir fumarate obtained by using a KBr pellet and Spectrum GX manufactured by Perkin-Elmer. Resolution was 4 cm⁻¹.

FIG. 28: X-ray powder diffraction pattern of polymorph II of valacyclovir fumarate according to the present invention obtained by using a Philips X'Pert PRO with CuKα radiation in 2θ=3-40° range.

FIG. 29: Typical DSC thermograph of polymorph II of valacyclovir fumarate obtained by using a DSC Pyris 1 manufactured by Perkin-Elmer. The experiment was done under a flow of nitrogen (35 ml/min) and heating rate was 10° C./min. A standard sample pan was used.

FIG. 30: Typical TGA thermograph of polymorph II of valacyclovir fumarate obtained by using thermogravimetric analysis (TGA) using TGA 7 manufactured by PerkinElmer. The experiments were done under flow of nitrogen (35 ml/min) and heating rate was 10° C./min.

FIG. 31: FTIR pattern of polymorph II of valacyclovir fumarate obtained by using a KBr pellet and Spectrum GX manufactured by Perkin-Elmer. Resolution was 4 cm⁻¹.

FIG. 32: X-ray powder diffraction pattern of polymorph I of valacyclovir tartrate according to the present invention obtained by using a Philips X'Pert PRO with CuKα radiation in 2θ=3-40° range.

FIG. 33: FTIR pattern of polymorph I of valacyclovir tartrate obtained by using a KBr pellet and Spectrum GX manufactured by Perkin-Elmer. Resolution was 4 cm⁻¹.

FIG. 34: X-ray powder diffraction pattern of polymorph I of valacyclovir citrate according to the present invention obtained by using a Philips X'Pert PRO with CuKα radiation in 2θ=3-40° range.

FIG. 35: Typical DSC thermograph of polymorph I of valacyclovir citrate obtained by using a DSC Pyris 1 manufactured by Perkin-Elmer. The experiment was done under a flow of nitrogen (35 ml/min) and heating rate was 10° C./min. A standard sample pan was used.

FIG. 36: Typical TGA thermograph of polymorph I of valacyclovir citrate obtained by using thermogravimetric analysis (TGA) using TGA 7 manufactured by Perkin-Elmer. The experiments were done under flow of nitrogen (35 ml/min) and heating rate was 10° C./min.

FIG. 37: FTIR pattern of polymorph I of valacyclovir citrate obtained by using a KBr pellet and Spectrum GX manufactured by Perkin-Elmer. Resolution was 4 cm⁻¹.

FIG. 38: X-ray powder diffraction pattern of valacyclovir base obtained by using a Philips X'Pert PRO with CuKα radiation in 2θ=3-40° range.

FIG. 39: Typical DSC thermograph of valacyclovir base obtained by using a DSC Pyris 1 manufactured by Perkin-Elmer. The experiment was done under a flow of nitrogen (35 ml/min) and heating rate was 10° C./min. A standard sample pan was used.

FIG. 40: Typical TGA thermograph of valacyclovir base obtained by using thermogravimetric analysis (TGA) using TGA 7 manufactured by Perkin-Elmer. The experiments were done under flow of nitrogen (35 ml/min) and heating rate was 10° C./min.

FIG. 41: FTIR pattern of valacyclovir base obtained by using a KBr pellet and Spectrum GX manufactured by Perkin-Elmer. Resolution was 4 cm⁻¹.

FIG. 42: Typical DVS isotherm plot of valacyclovir base

### EXAMPLES

### Example 1

### Preparation of Valacyclovir Free Base

Valacyclovir hydrochloride hydrate (13 mmol) was suspended in methanol (50 mL) and a solution of NaOH (0.6 g; 15 mmol) in methanol (18 mL) was added drop wise to the suspension of valacyclovir salt. The reaction mixture was stirred for about 2 hours at room temperature. The resulting precipitate was filtered.

### Example 2a

### Preparation of Valacyclovir Mesylate Form I

Valacyclovir base (6.0 g; 18.50 mmol) was suspended in ethanol (50 mL) and heated at reflux. Methanesulfonic acid, anhydrous (1.4 mL; 21.56 mmol) was dissolved in ethanol (30 mL) and added drop wise into the suspension of valacyclovir base, resulting in dissolution. The heating of the solution was discontinued and the reaction mixture was stirred overnight (about 15 h). The reaction mixture was cooled to about 0° C. and stirred for about 2 hours. The resulting precipitate was filtered and dried in a vacuum oven at 85° C., yielding 6.72 g of valacyclovir mesylate form I.

### Example 2b

### Preparation of Valacyclovir Mesylate Form I

Valacyclovir base (500 mg; 1.54 mmol) was suspended in methanol (10 mL) and heated to about 65° C. Methanesulfonic acid, anhydrous (0.11 mL; 1.69 mmol) was dissolved in methanol (5 mL) and added drop wise into the suspension of valacyclovir base, resulting in dissolution. Heating of the solution was discontinued and the reaction mixture was stirred until precipitation. The solid was filtered, yielding 60 mg of valacyclovir mesylate.

### Example 3

### Preparation of Valacyclovir Phosphate Form I

Valacyclovir base (500 mg; 1.54 mmol) was suspended in absolute ethanol (10 mL) and heated at about 85° C. Phosphoric acid, min. 85% (0.114 mL, 1.69 mmol) was dissolved in absolute ethanol (5 mL) and added drop wise into the suspension of valacyclovir base. Additional absolute ethanol (10 mL) was added to the dense suspension of valacyclovir base. Heating was discontinued and the reaction mixture was stirred for about 3 hours at room temperature. The resulting precipitate was filtered and washed with ethanol, yielding 530 mg of valacyclovir phosphate form I.

### Example 4

### Preparation of Valacyclovir Phosphate Form II

Valacyclovir phosphate (30 mg; 0.07 mmol) was dissolved in water and methanol (the total volume of solvent was 2 mL consisting of varying ratios of water and methanol) and the solution was left to stand in an open flask at room temperature in order to crystallize. The solid was filtered to yield valacyclovir phosphate form II.

The experiment was repeated using ethanol, 1-propanol, 2-propanol, acetonitrile, benzonitrile or benzyl alcohol instead of methanol.

### Example 5

### Preparation of Valacyclovir Phosphate Form III

Valacyclovir phosphate form II was heated in a vacuum oven at 85° C. for about 18 hours giving rise to valacyclovir phosphate form III.

### Example 6

### Preparation of Valacyclovir Maleate Form I

Valacyclovir base (500 mg; 1.54 mmol) was suspended in ethanol, p.a. (10 mL) and heated to about 85° C. Maleic acid (180 mg, 1.55 mmol) was dissolved in ethanol, p.a. (10 mL) and added drop wise into the suspension or valacyclovir base, resulting in dissolution. Heating was discontinued and the reaction mixture was stirred for about 3 hours at room temperature. The resulting precipitate was filtered, washed with ether and dried in a vacuum oven at 65° C. for 4 h and re-crystallized from water/acetonitrile mixture, giving rise to valacyclovir maleate form I.

### Example 7

### Preparation of Valacyclovir Fumarate Form I

Valacyclovir base (1.0 g; 3.08 mmol) was suspended in ethanol, p.a. (20 mL) and heated at about 85° C. Fumaric acid (182 mg, 1.56 mmol) was dissolved in ethanol, p.a. (20 mL) and added drop wise to the suspension of valacyclovir base. The reaction mixture was stirred for about 1 hour at 85° C. The heating was discontinued and the reaction mixture was stirred for an additional 2 hours. The resulting precipitate was filtered, washed with ethanol and dried in a vacuum oven at 85° C. for 24 hours, yielding 1.09 g of valacyclovir fumarate form I.

### Example 8

### Preparation of Valacyclovir Fumarate Form II

Valacyclovir fumarate (30 mg; 0.08 mmol) was dissolved in water and 1-propanol (the total volume of solvent was 2 mL, consisting of varying ratios of water and 1-propanol) and the solution was left to stand in a sealed flask at room temperature to crystallize, yielding valacyclovir fumarate form II.

The experiment was repeated using 2-PrOH, acetonitrile or benzyl alcohol instead of 1-PrOH.

### Example 9

### Preparation of Valacyclovir Tartrate Form I

Valacyclovir base (500 mg; 1.54 mmol) was suspended in absolute ethanol (20 mL) and heated at 85° C. Tartaric acid (116 mg, 0.77 mmol) was dissolved in absolute ethanol (20 mL) and added drop wise into the suspension of valacyclovir base. The heating was discontinued and the reaction mixture was stirred over night. The resulting precipitate was filtered, washed with ethanol and dried in a vacuum oven at 85° C. for 3 hours, yielding 510 mg of valacyclovir tartrate form I.

### Example 10

### Preparation of Valacyclovir Citrate Form I

Valacyclovir base (1.0 g; 3.08 mmol) was suspended in methanol, p.a. (20 mL) and heated at about 75° C. Citric acid monohydrate (640 mg, 1.56 mmol) was dissolved in methanol, p.a. (20 mL) and dried on molecular sieves for about 15 minutes. The solution of citric acid was added drop wise to the suspension of valacyclovir base, resulting in complete dissolution. The reaction mixture was stirred for about 2 hours at 75° C. The heating was discontinued and the reaction mixture was stirred for an additional 2 hours. The resulting precipitate was filtered and dried at room temperature for about 20 hours, yielding 944 mg of valacyclovir citrate form I.

1. A pharmaceutically acceptable salt of valacyclovir, wherein said salt is formed between valacyclovir free base and a pharmaceutically acceptable acid selected from the group consisting of methanesulphonic acid, phosphoric acid, maleic acid, fumaric acid, tartaric acid and citric acid.

2. The salt of claim 1 wherein the salt is valacyclovir mesylate.

3. The salt of claim 1 wherein the salt is valacyclovir phosphate.

4. The salt of claim 1 wherein the salt is valacyclovir maleate.

5. The salt of claim 1 wherein the salt is valacyclovir fumarate.

6. The salt of claim 1 wherein the salt is valacyclovir tartrate.

7. The salt of claim 1 wherein the salt is valacyclovir citrate.

8.-99. (canceled)

100. A pharmaceutical composition comprising a therapeutically effective dose of a valacyclovir salt according to claim 1, or a polymorphic form thereof, together with a pharmaceutically acceptable carrier, diluent or excipient therefor.

101.-104. (canceled)

105. A method of treating a disease state prevented, ameliorated or eliminated by the administration of a compound having anti-viral activity, to a patient in need of such treatment, which comprises administering to the patient a therapeutically effective amount of a valacyclovir salt according to claim 1.

106. A method according to claim 105, wherein the disease state is caused by a viral infection.

107. A method according to claim 105, wherein said disease state is caused by a herpes viral infection.

108.-110. (canceled)
